(19) United States
(12) Patent Application Publication
(10) Pub. No.: US 2007/0031822 A1
(43) Pub. Date: Feb. 8, 2007

(54) Ndr kinase modulators

(76) Inventors: Eric Devroe, Cambridge, MA (US); Alan Engelman, Brookline, MA (US); Pamela Silver, Cambridge, MA (US)

Correspondence Address: FISH & RICHARDSON PC, P.O. BOX 1022, MINNEAPOLIS, MN, 55440-1022, US

(21) Appl. No.: 10/542,517

(22) PCT Filed: Jan. 22, 2004
(86) PCT No.: PCT/US04/01679

§ 371 Date: Apr. 14, 2006

Related U.S. Application Data
(60) Provisional application No. 60/441,898, filed on Jan. 22, 2003.

(51) Int. Cl. C12Q 1/70; C12Q 1/68; C12Q 1/48

(52) U.S. Cl. 435/5; 435/6; 435/15

(57) ABSTRACT

Methods of identifying agents that modulate NDR kinases, including those that modulate NDR1 and NDR2 kinases and methods of using those agents to inhibit retroviral pathogenesis are among the methods described herein.

### CROSS-REFERENCE TO RELATED APPLICATIONS

This application claims the benefit of the filing date of U.S. Ser. No. 60/441,898, filed Jan. 22, 2003, the contents of which are hereby incorporated by reference in their entirety.

The work described herein was funded, in part, by a grant from the National Institutes of Health (Grant No. AI39394 awarded to Alan Engelman). The United States government may, therefore, have certain rights in the invention.

### FIELD OF THE INVENTION

The present invention relates generally to methods of identifying agents that modulate kinase activity, including those that act as agonists or antagonists of an NDR kinase, and to methods of using those agents to inhibit viral pathogenesis.

### BACKGROUND

Many fundamental cellular events are mediated by reversible protein phosphorylation. Whether or not a protein is active can depend on whether or not it is phosphorylated, and the activities of specific target proteins can be regulated by the opposing actions of protein kinases and protein phosphatases. Generally, these enzymes are specific for either serine/threonine or tyrosine phosphoacceptors, although some enzymes have dual specificity. The fact that many kinases, phosphatases, and the signal transduction pathways in which they participate have been highly conserved during the course of evolution belies their importance. In recent years, many investigators have focused on the mechanisms by which protein phosphorylation controls the cell cycle; a number of cellular protooncogenes encode members of the serine/threonine kinase family and it has become increasingly clear that kinases within this family are key components of the cell cycle regulatory network. Completely delineating these complex pathways should help our understanding of oncogenesis and tumor progression.

The NDR (nuclear, Dbf-2-related) kinases, including NDR1 and NDR2, are thought to be ubiquitously expressed serine/threonine protein kinases that are homologous to the Dbf2 kinase of Saccharomyces cerevisiae (Millward et al., Proc. Natl. Acad. Sci. USA 92:5022-5026, 1995; U.S. Pat. No. 5,981,205). C. elegans, Drosophila, and human NDR kinases have been identified, and the Drosophila and human proteins are about 68% identical (Millward, supra). The NDR1 and NDR2 amino acid sequences each contain all of the 12 protein kinase catalytic subdomains identified by Hanks and Quinn (Meth. Enzymol. 200:38-62, 1991). NDR1 also contains a short basic peptide (KRKAETWKRNRR; SEQ ID NO: 5), which contains a calmodulin binding domain and is believed to be responsible for nuclear accumulation.

### SUMMARY

Generally, the compositions and methods described herein relate to NDR kinases, agents that modulate NDR kinase activity (i.e., agonists, which stimulate, and antagonists, which inhibit, NDR kinases), assays by which such agents can be identified, compositions containing them, and methods of using them. As described further below, agents identified by the methods of the present invention can be incorporated in pharmaceutically acceptable compositions for the treatment of viral (e.g., retroviral) infections. Accordingly, the invention features the use of the NDR kinase inhibitors described herein, alone or in combination with known NDR kinase inhibitors, for the preparation of medicaments.

More specifically, the invention features methods of identifying an agent that modulates (e.g., inhibits or stimulates) an NDR kinase (e.g., NDR1 or NDR2) and/or inhibits retroviruses. While various embodiments are described below, we note here that the method can include incubating an NDR kinase with a potential modulating agent under conditions (e.g., conditions at or near a physiological temperature and pH) that permit the agent to modulate the kinase and performing an assay to determine the level of expression or activity of the NDR kinase. A change in the level of kinase expression or activity (e.g., relative to a control or reference value, which we may also refer to as a “standard”) indicates that the agent is a modulator of the NDR kinase. Where the agent increases expression or activity of the NDR kinase, the agent agonizes (or stimulates) the kinase. Where the agent decreases activity or expression of the NDR kinase (e.g., NDR1, NDR2), the agent antagonizes (or inhibits) the kinase.

One can carry out the screening methods by providing the agent or a collection of agents (e.g., a library) and providing an NDR kinase (e.g. NDR1 or NDR2) or an NDR kinase-expressing cell (as described further below, that cell may also be one that is infected with a retrovirus). The cell can be one that naturally expresses an NDR kinase or one that has been genetically engineered to express or overexpress the kinase. Moreover, the cell and/or the kinase can be mammalian (e.g., the cell can be a human cell and the kinase can be human NDR1 or NDR2). Depending on the configuration of the assay, the NDR kinase can be substantially pure (e.g., at least about 80% (e.g., 80-85, 85-90, 90-95, or 95-99%) pure when assessed in vitro) or contained within a biological sample such as a fluid sample (e.g., a blood sample), cellular lysate or whole cell or tissue (the assays of the invention can be carried out in cell culture or cells can be exposed to a potential modulatory agent in vivo).

The assays to identify an agent as an NDR kinase modulator can include assessing the level of NDR kinase mRNA (e.g., by performing a Northern blot or other quantitative or semi-quantitative procedure, such as those in which PCR is used to amplify nucleic acids that encode an NDR kinase) or the level of NDR protein expression (e.g., by assessing a Western blot or performing another antibody-based quantitative or semi-quantitative method).

When assessing activity, the assay can include assessing the degree to which an NDR kinase substrate has been phosphorylated (e.g., by Western blot, or ELISA). Examples of NDR kinase substrates include polypeptides having the amino acid sequence KKRNRRLSVA (SEQ ID NO:6, histone H1, and myelin basic protein (MBP). NDR kinases also autophosphorylate.

In other embodiments, the methods (e.g., the screening assays) of the invention can identify modulators of an NDR kinase by determining whether a putative modulator disrupts or facilitates the formation of a complex that includes an NDR kinase and a second protein (e.g., a heterologous protein, which may or may not naturally interact with the kinase). For example, one can assess complexes between the NDR kinase and a calcium binding protein (e.g., an EF-hand containing calcium binding protein such as, for example, S100B or S100). In addition to, or instead of, examining the formation of complexes with calcium binding proteins, one can assess complexes that include an NDR kinase and a Mob protein (e.g., a Mob 2 protein, also known as HCCA2, found under GenBank Acc. No. NP_{—}443731, GI No: L34594669; a Mob4A protein, also known as Mob1B, the sequence of which can be found under GenBank Acc. No. NP_{—}775739, GI No. 27735029; a MOB-LAK protein, found under GenBank Acc. No. NP_{—}570719, GI No. 18677731) or a variant thereof (e.g., a protein at least 50% identical to a Mob protein). Mob proteins can activate NDR kinases. Thus, a modulator that inhibits interaction between an NDR kinase and a Mob protein can, in turn, inhibit the kinase by preventing its activation. A modulator that enhances interaction between an NDR kinase and a Mob protein can agonize the kinase.

The assays of the invention can also be configured to assess characteristics that are associated with the subject kinase other than expression, activity, or complex formation. These characteristics include the extent to which the kinase interacts with a retrovirus (e.g., the extent to which it is cleaved by retroviral proteins) and its subcellular location. Based on our discovery of the interaction between NDR kinases and retroviruses (described further below), the assays of the invention can also be carried out with virally infected (e.g., retrovirally infected) cells. For example, one can provide a retrovirus-infected cell; provide a potential kinase modulator (i.e. an “agent” or “test agent” that may stimulate or inhibit an NDR kinase); contact the cell with the modulator; and determine whether the virions produced by the cell, relative to control (or to a standard or reference), exhibit altered pathogenicity, altered infectivity, package more or less NDR kinase, contain a viral protein that is more or less phosphorylated, contain a host protein that is more or less phosphorylated, or exhibit altered reverse transcriptase activity. An assay to determine whether virions produced by the cell exhibit altered pathogenicity can include determining the extent to which cells infected with the virions survive (e.g., the percentage survival in a given population). For example, cell survival can be evaluated at 2, 3, 4, 5, 6, 7, 8, 9, 10, 11, 12, 13, 14, or more days following infection and/or exposure to the test agent.

As described further in the examples below, we have discovered that an NDR kinase is susceptible to cleavage by a protease such as a retroviral protease (e.g., an HIV-1 protease). Accordingly, one can examine the effect of an agent on the ability of a protease to cleave the NDR kinase. Agents that facilitate the cleavage are potential inhibitors of the kinase.

In some embodiments, the modulator may partially inhibit an activity of retroviruses produced by the cell. For example, the modulator may have a desirable effect by decreasing cytopathogenicity or infectivity of virions. Decreasing cytopathogenicity of retroviruses produced by the cell can promote survival of the cell. In some embodiments, the modulator may partially agonize an activity of retroviruses produced by the cell. For example, increasing cytopathogenicity can promote death of infected cells, thereby promoting elimination of reservoirs of persistently infected cells in an individual. The modulator can affect a characteristic of the retroviruses (e.g., infectivity or cytopathogenicity) in vitro, and/or in vivo. As cytopathogenicity of virions in T cells and/or macrophages can be altered, these cell types may be specifically employed in the methods of the invention.

Given our discoveries of the interaction between NDR kinases and retroviruses, the methods of the invention can determine whether a modulator of an NDR kinase also modulates a retrovirus. For example, one can: (a) incubate an identified or suspected modulator of an NDR kinase with a retrovirus-infected cell under conditions that permit the modulator to affect the retroviruses produced by the cell, and (b) perform an assay to evaluate a characteristic of the retrovirus. A change in the characteristic of the retrovirus(es) (e.g., relative to a control, or a reference value) indicates that the modulator of the NDR kinase also modulates the retrovirus. As noted in connection with the assays described above, the agent can inhibit the characteristic (e.g., inhibit infectivity or cytopathogenicity) of the retrovirus or enhance it (e.g., the test agent can increase cytopathogenicity of the retrovirus. Any of the methods described herein by which agents are tested as modulators of an NDR kinase can be performed prior to assessing the effect of the agent on a retrovirus. For example, one can contact the NDR kinase with an agent under conditions that permit the agent to modulate the kinase and determine whether an activity of the kinase is changed in the presence of the agent, relative to a control or a reference value.

In specific embodiments, one can evaluate a characteristic of the retrovirus by performing an assay for infectivity, viability, cytopathogenicity, or any parameter that correlates with infectivity or viability. For example, the method can include: (a) transfecting a producer cell with one or more nucleic acids (e.g., plasmids) that contain a functional HIV genome; (b) incubating the producer cell with the NDR protein kinase inhibitor under conditions that permit the inhibitor to inhibit the NDR protein kinase; and (c) maintaining the producer cell under conditions that allow the production of HIV virions. A change in the characteristic of the virions, relative to control, indicates that the modulator of the NDR protein kinase is also a modulator of the retrovirus.

In other specific embodiments, the producer cell can be transfected with two nucleic acids, the first of which (i) lacks a functional envelope gene and (ii) includes a reporter gene (e.g., a gene encoding a fluorescent marker such as luciferase) in place of an HIV gene (e.g., Nef, Vpr, or Vif), and the second of which contains the envelope gene (Env). Cytopathogenicity or infectivity of the virions can be evaluated in standard assays. For example, the assay can further include infecting a naïve cell with the virions, wherein the naïve cell is permissive for HIV infection (e.g., a cell that expresses a receptor for HIV, such as CD4). Many cell types, including HeLa cells, can be used to produce virions. Where a cell that is permissive for HIV infection is used, the cell can be a HeLa cell that expresses a CD4 gene.

The assay can include determining the activity of the reporter gene in the naïve cell, relative to the activity of the reporter gene from virions produced in a control cell (e.g., a producer cell in which virions were produced in the absence of the NDR protein kinase inhibitor).

The reporter gene can be any gene the expression of which can be determined. Exemplary reporter genes include genes that encode enzymes whose activity can be measured (e.g., β-galactosidase or chloramphenicol acetyltransferase) and genes that encode light-emitting proteins, such as the green fluorescent protein (GFP) or luciferase.

The assay to identify an anti-retroviral agent can also be carried out by determining whether the virions produced in a cell exposed to the inhibitor package less NDR kinase relative to control; determining whether the virions contain a viral protein that is phosphorylated to a lesser extent than virions produced in a control cell; or determining whether the virions exhibit less reverse transcriptase activity than virions produced in a control cell.

Wherever a cell or cell-based assay is described herein, it is expected that a plurality of cells, rather than a single cell, can be employed.

Modulatory agents that inhibit NDR1 or stimulate NDR2 enhance the survival of cells infected with a retrovirus. Modulatory agents that stimulate NDR1 or inhibit NDR2 decrease the survival of cells infected with a retrovirus. It may be desirable to enhance survival of infected cells in situation in which a patient exhibits a low CD4⁺ T cell count (e.g., below 500, 200, or 100 CD4⁺ T cells per microliter in a blood or PBMC sample). Improved survival of infected cells, such as infected T cells, may prevent a decline, and may even enhance, immune function in an individual. Alternatively, it may be desirable to decrease survival of infected cells, e.g., in clinical stages of infection characterized by high levels of viral replication. Reducing survival of infected cells may interfere with viral replication and thereby suppress continuous re-infection of cells by virus produced in the body. It may also be desirable in eliminating reservoirs of infected cells which are otherwise inaccessible to host immune mediators or other anti-viral agents. One can evaluate which type of agent is appropriate by various means. For example, NDR modulators can be tested with cell types known to be persistently infected with virus to determine whether the agent has the desired effect. The impact of decreased cell survival on viral production can be measured with in vitro assays, and/or by determining viral load in vivo. The effector function (e.g., antigen-induced cytokine production) of infected T cells in the presence of an NDR modulator can indicate whether or not infected cells with increased survival are also functional.

Where a retrovirus is employed, that retrovirus can be the human immunodeficiency virus-1 (HIV-1), the human immunodeficiency virus-2 (HIV-2), the human T cell leukemia virus-1 (HTLV-1), the human T cell leukemia virus-2 (HTLV-2), the simian immunodeficiency virus (SIV), the feline immunodeficiency virus (FIV), or the equine infectious anemia virus (EIAV). The retrovirus can also be an endogenous retrovirus.

Alternatively, or in addition, the methods of the invention can include examining the subcellular location of the kinase. Agents that alter the position of the kinase within the cell will alter its ability to function and, therefore, are potential kinase modulators.

The assays of the invention can be configured to assess any type of agent. For example, the test agent can be a small molecule, a peptide, or a nucleic acid. The nucleic acid can be, for example, a DNA, RNA or hybrid nucleic acid, and it can be complementary to all or to a portion of the sequence encoding an NDR kinase. As described further below, the nucleic acid can be a double-stranded nucleic acid, a small interfering RNA (siRNA), or any nucleic acid that mediates RNA interference (RNAi).

Optionally, any of the assays of the invention can be run in parallel with a control assay or can include a step in which the results are compared with a standard or reference point (e.g., a standard amount of kinase expression or activity, a standard degree of complex formation; a standard amount of susceptibility to cleavage; or a typical subcellular locale). Those of ordinary skill in the art are well able to select appropriate control parameters. For example, it is usual to carry out control experiments in which the conditions are essentially the same as those of the assay conditions except that the agent is omitted or supplied in an inert form. Such controls could certainly be performed in the context of the present invention.

The methods (e.g., screening assays) to identify an agent that modulates an NDR kinase can further include a step of producing the identified agent, and any identified agent may be produced in sufficient quantities to perform additional assays (e.g., an agent identified in an in vitro or cell culture assay may be produced in sufficient quantities to carry out in vivo studies in laboratory animals or human patients).

In another aspect, the invention features methods of treating a subject who has been, or who is at risk of being, exposed to a retrovirus (e.g., a lentivirus). The method can include, for example, administering to the subject an effective amount of a modulator (e.g., a nucleic acid that mediates RNA interference (RNAi)) of an NDR kinase (e.g., NDR1, NDR2). The retrovirus can be, for example, HIV-1, HIV-2, HTLV-1, HTLV-2, SIV, FIV, or EIAV.

A modulator that is a nucleic acid can be optimally contained within an expression vector, and such vectors can be used, for example, to reduce the quantity of NDR kinase in the host cell or to interfere with the ability of the NDR kinase to form a complex with retroviral proteins (e.g., retroviral proteases) and/or retroviral capsids. The inhibitor may reduce the catalytic activity of the NDR kinase. The inhibitor can reduce cytopathogenicity of the virus.

Agents that inhibit NDR kinases can be administered to patients (e.g., patient who have been diagnosed as having a retroviral infection) in combination with at least one other anti-retroviral agent (e.g., a reverse transcriptase inhibitor, a viral protease inhibitor, or a viral entry inhibitor). Examples of anti-retroviral agents include zidovudine (AZT), lamivudine (3TC), didanosine (ddI), abacivir, zalcitabine (ddC), stavudine (d4T), tenofovir disproxil fumarate (DF), efavirenz, rescriptor, viviradine, nevirapine, delaviridine, saquinavir, ritonavir, indinavir, nelfinavir, agenerase, viracept, amprenavir, lopinavir, enfuviritide and hydroxyurea.

The details of one or more embodiments of the invention are set forth in the accompanying drawings and the description below. Other features, objects, and advantages of the invention will be apparent from the description and drawings, and from the claims. In accordance with the rules governing U.S. patent prosecution, all cited patents, patent applications, and references (including references to public sequence database entries) are incorporated by reference in their entireties for all purposes.

### DESCRIPTION OF DRAWINGS

FIG. 1 is a representation of the nucleic acid sequence of a human NDR1 kinase cDNA (SEQ ID NO: 1; see also Genbank Accession number NM_{—}007271 and Genbank GI No. 6005813).

FIG. 2 is a representation of the amino acid sequence of a human NDR1 kinase (SEQ ID NO:2; see also Genbank Accession No. NP_{—}009202, Genbank GI No. 6005814). The potential PR cleavage site is located at amino acid position 440 in NDR1.

FIG. 3. is a representation of the nucleic acid sequence of a human NDR2 kinase cDNA (SEQ ID NO:3; see also Genbank Accession No. NM_{—}015000 and Genbank GI No. 24307970). FIG. 3A depicts nucleotides 1-3000 of SEQ ID NO:3. FIG. 3B depicts nucleotides 3001-4725 of SEQ ID NO:3.

FIG. 4 is a representation of the amino acid sequence of a human NDR2 kinase (SEQ ID NO:4; see also Genbank Accession No. NP_{—}055815 and Genbank GI No. 24307971). The potential PR cleavage site is located at amino acid position 439 in NDR2.

FIG. 5 is an alignment of human NDR1 and NDR2 amino acid sequences. The PR cleavage site is indicated.

FIG. 6A is a schematic diagram depicting the steps used to construct cells in which NDR2 was knocked down (NDR2^{KD}), cells in which NDR1 was knocked down (NDR1^{KD}), and cells in which both NDR1 and NDR2 were knocked down (NDR1^{KD}/NDR2^{KD}).

FIG. 6B is a graph and picture depicting the level of NDR1 and NDR2 mRNA (graph, upper panel), and NDR1 and CDK4 protein (pictures of Western blot, lower panels) expressed by control cells, NDR2^{KD} cells, NDR1^{KD} cells, and NDR1^{KD}/NDR2^{KD} cells.

FIG. 6C is a set of pictures depicting control cells, NDR2^{KD} cells, NDR1^{KD} cells, and NDR1^{KD}/NDR2^{KD} cells at 9 days post-infection with HIV-1 (9 dpi) and 10 days post-infection with HIV-1 (10 dpi) at 100× and 40× magnification, respectively. Next to each set of pictures is a set of graphs depicting DNA content vs. cell number for control cells, NDR2^{KD} cells, NDR1^{KD} cells, and NDR1^{KD}/NDR2^{KD} cells which were mock-infected, or at 9 days post-infection with HIV-1 (9 dpi).

FIG. 6D is a graph depicting viral particle release into culture supernatants, as measured by reverse transcriptase (RT) activity 1-11 days post infection with HIV-1. Filled triangles correspond to NDR1^{KD} cells. Filled diamonds correspond to NDR1^{KD} cells. Xs correspond to NDR2^{KD} cells. Filled squares correspond to NDR1^{KD}/NDR2^{KD} cells.

### DETAILED DESCRIPTION

The present invention is based, in part, on our discovery that NDR kinases are involved in retroviral cytopathogenicity. We identified NDR1 as a component of HIV-1 particles, and found that the kinase was also packaged into HIV-2, HTLV-1, SIVmac, and EIAV virions. We determined that the highly related kinase, NDR2, is also incorporated in HIV-1 particles. In addition, we determined that the HIV-1 protease (PR) proteolytically processes the C-terminus of both NDR1 and NDR2, both in virions and within infected producer cells. We have discovered that cells with reduced amounts of NDR1 are refractory to the cytopathic effects of HIV-1. Conversely, cells that have reduced amounts of NDR2 are hypersensitive to the cytopathic effects of HIV-1. Thus, the cytopathogenicity of HIV-1 can be directly modulated by the NDR1 and NDR2 kinases.

The findings described here have important implications for the management and treatment of patients infected with a retrovirus (e.g., HIV-1). Inhibition of NDR1 and/or activation of NDR2 can promote cell survival of cells susceptible to the pathogenic effects of HIV, such as infected T cells. Promotion of cell survival can enhance immune system function in spite of persistent viral replication. Conversely, inhibition of NDR2 and/or activation of NDR1 can promote death of infected cells. This can be applied to destroy refractory viral reservoirs by promoting cell death of HIV-1 infected cells. Accordingly, the methods of the invention include those in which NDR1 and/or NDR2 kinase inhibitors and agonists are identified and, if desired, further tested for their ability to inhibit retroviral pathogenesis. Of course, NDR1 or NDR2 kinase inhibitors or agonists previously identified can also be used to inhibit retroviral pathogenesis (this is a new use based on our studies), and these inhibitors can be administered to treat patients who are suffering from a disease or condition associated with retroviral infection. Thus, methods in which an NDR1 or NDR2 kinase modulator is administered to a patient are within the scope of this invention, whether the inhibitors or agonists are newly identified by the present methods or previously known.

NDR1 and NDR2 kinases are characterized by a number of structural features, which can be exploited in the methods of the invention (e.g., the sequences mentioned here, and others, can be targeted by a potential inhibitor). The presence of a DIKPDN (SEQ ID NO:7) amino acid sequence in the catalytic subdomain VIb (residues 206-221 in NDR1) and a GTPDYIAPE (SEQ ID NO:8) sequence in subdomain vm (residues 277-294 in NDR1) of human NDR1 and NDR2 indicates that these kinases can have serine/threonine specificity (Millward et al., Proc. Natl. Acad. Sci. USA 92:5022-5026, 1995). NDR1 and NDR2 have an unusual catalytic subdomain structure, in that two catalytic subdomains (VII and VIM that are contiguous in the primary structure of most other protein kinases are separated by about 30 amino acids in NDR1 and NDR2 (at residues 244 to 276 of NDR1; Millward et al., supra). NDR1 and NDR2 do not have domains homologous to Src homology-2 domains (SH2), Src homology-3 domains (SH3), or Pleckstrin homology domains (Millward et al., supra). Recombinant human NDR1 kinase does not phosphorylate nonspecific kinase substrates, such as histone H1, myelin basic protein, casein, and phosvitin in in vitro kinase assays, but does exhibit autophosphorylation (Millward et al., supra). NDR2 phosphorylates histone H1 and myelin basic protein. Deletion of amino acids 265-276 in the catalytic domain interferes with the nuclear localization of NDR1 Nillward et al., supra).

NDR1 can be activated by the calcium-responsive, EF-hand containing S100 proteins. Deletion of amino acids 65-81 of human NDR1 kinase results in reduced ability to bind S100 proteins. A peptide containing amino acids 62-84 of the NDR1 kinase inhibited calcium/S100-mediated activation of NDR1 (Millward et al., EMBO J. 17(20):5913-5922, 1998).

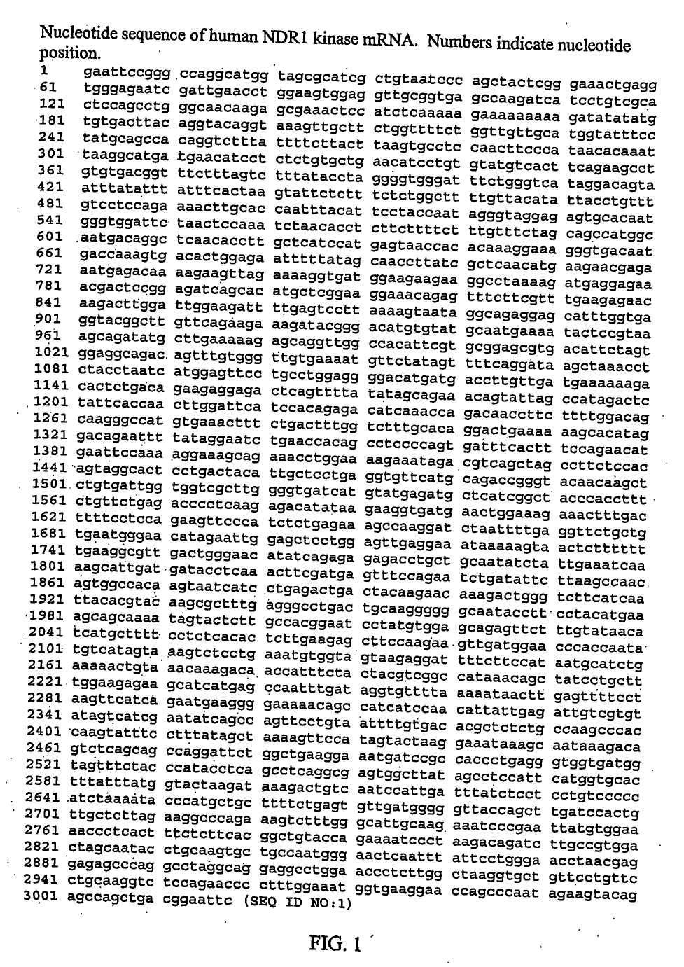

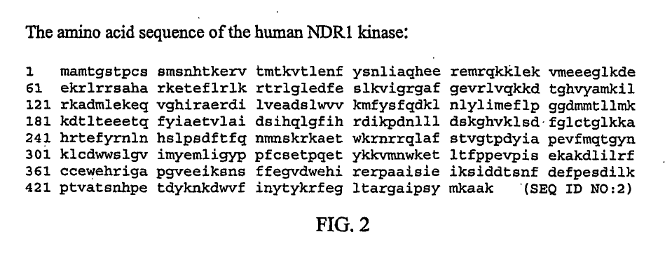

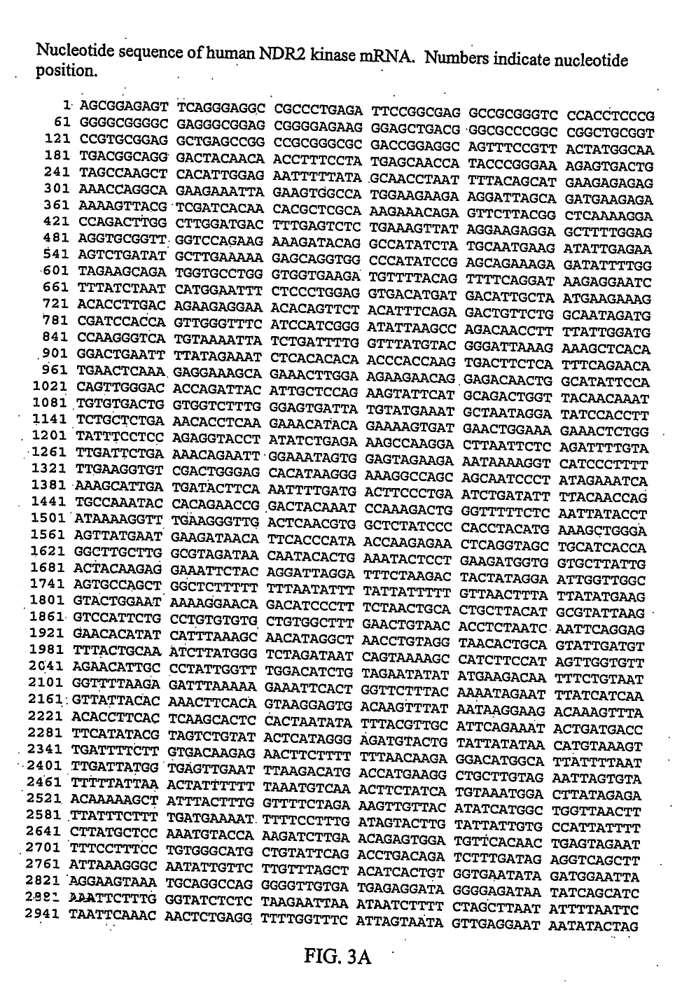

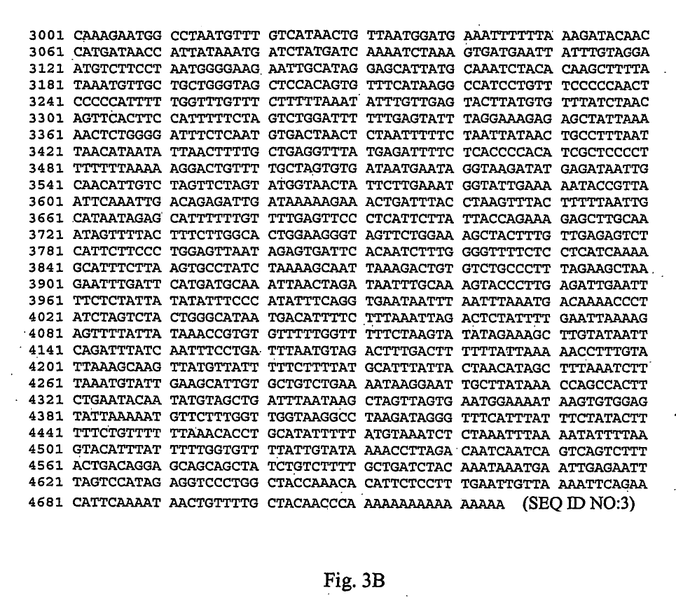

NDR kinases also can be activated by proteins of the Mob family. Mob family proteins are a group of highly conserved eukaryotic proteins that function as kinase-activating subunits. Structural and functional features of Mob proteins are described in Stavridi et al. (Structure, 11:1163-1170, 2003). Mob proteins contain the following conserved residues (numbered with respect to the amino acid sequence of human Mob 4A, found under GenBank Acc. No. NP_{—}775739, GI No. 27735029): P48, D52, W56, N69, M87, A89, A111, Y114, F112, P1113, Y163, F186 and F189 (see also FIG. 1 of Stavridi et al., supra). Interaction with Mob proteins is thought to occur via an N-terminal regulatory domain in NDR (Tamaskovic et al., FEBS, 546:73-80, 2003). NDR kinases are also potently activated by treatment with the protein phosphatase 2A inhibitor okadaic acid.

While we expect it will be more usual to carry out the methods of the invention with full-length NDR kinases, the invention is not so limited. Any of the assays described herein (see, for example, the following section) can be carried out with biologically active fragments or other mutants (e.g., mutants generated by substitution of one or more amino acid residues) of the NDR kinase (e.g., NDR1 or NDR2). The fragments or other mutants need not retain full biological activity; they need only retain sufficient biological activity to function in the screening assay.

Screening Assays. The invention encompasses methods, which may be referred to herein as “assays” or “screening assays,” that can be used to identify agents (or “modulators”) that bind to, or otherwise interact with, NDR1 and/or NDR2 protein kinases, the nucleic acids that encode them, and/or other biological materials with which they interact (e.g., enzymes, such as proteases, of viral proteins, or other viral molecules). The agents are referred to as modulators, as they may act as agonists, which stimulate, or antagonists, which inhibit an NDR kinase. As noted, the modulators (e.g., inhibitors) may interact directly with the NDR kinase, but the invention is not so limited. The methods of the invention may also identify modulators that interact with NDR kinases by way of binding to, or otherwise interfering with, molecules that act either upstream or downstream from the NDR1 or NDR2 kinase (i.e., molecules that participate in the biochemical pathway(s) that include an NDR1 or NDR2 kinase or viral proteins).

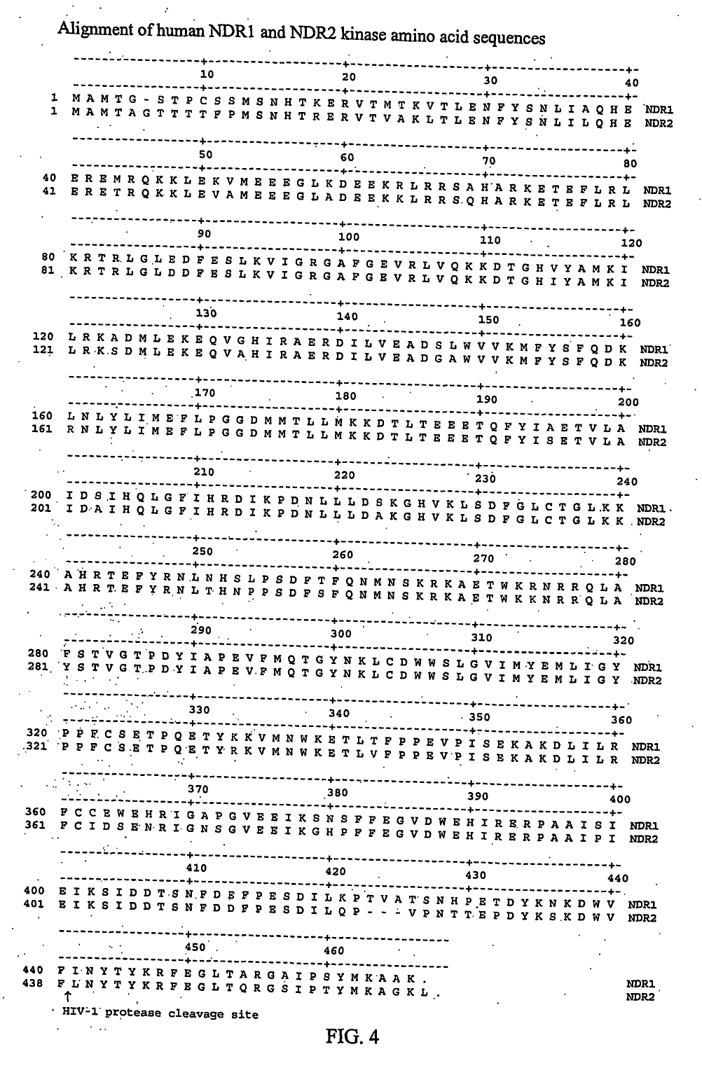

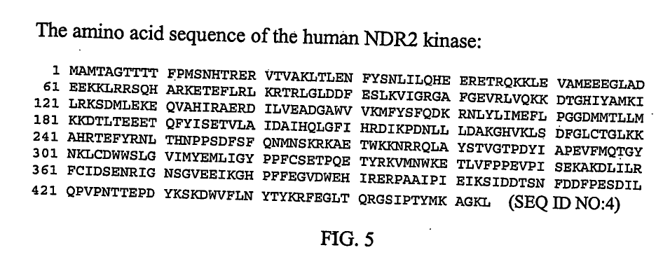

NDR1 and NDR2 kinases are approximately 87% identical at the amino acid level (see the alignment in FIG. 4). They can be distinguished using antibodies, by sequence analysis, by subcellular localization, or by indirect means (e.g. by determining an activity specific for one of the kinases).

While we discuss potential inhibitors and agonists below, we note here that the agents can be essentially any physiologically acceptable (i.e., non-lethal) substance. For example, an inhibitor can be a protein, peptide, or polypeptide (all of these terms refer to linear polymers of amino acid residues, regardless of glycosylation or other post-translational modification; the term “protein” being commonly used to refer to full-length, naturally occurring proteins and the terms “peptide” or “polypeptide” being commonly used to refer to fragments thereof). The NDR inhibitor or agonist can also be a peptidomimetic, a peptoid, another small molecule (e.g., a small synthetic molecule), a nucleic acid, or another drug. While the invention is not limited to agents that act by any particular mechanism, some of these agents (e.g., anti-NDR antibodies or fragments thereof (including single-chain antibodies)) may inhibit the activity of the NDR kinase, while others (e.g., an antisense oligonucleotide or a siRNA) can alter NDR1 or NDR2 kinase expression. Likewise, an inhibitor can affect the expression or activity of a molecule that acts on NDR1 or NDR2 kinase (e.g., the calmodulin-related polypeptides S100B and S100 noted above, both of which are thought to activate NDR kinase by binding the N-terminal region of the kinase; U.S. Pat. No. 6,528,776; Millward et al., EMBO J. 17:5913-5922, 1998) or upon which an NDR kinase acts (e.g., a protein NDR1 or NDR2 phosphorylates; an NDR kinase substrate). Other inhibitors can affect the translocation of an NDR protein from one region of a cell (e.g., the cytoplasm) to another (e.g., the nucleus). Agents identified as inhibitors can be used to modulate the expression or activity of an NDR kinase in a therapeutic protocol. They can, for example, disrupt the events that normally occur when an NDR kinase interacts with some component of a retrovirus (e.g., retrovirus virions, structural proteins, or enzymes).

The assays used to identify NDR1 or NDR2 kinase modulators (whether inhibitors or stimulatory agents) can be carried out variously in vitro, in cell culture, or in vivo, and they can reveal the presence or absence of NDR1 or NDR2 kinase (i.e., they can be qualitative) or the level of its expression or activity (i.e., they can be quantitative). Moreover, the assays can be conducted in a heterogeneous format (where an NDR kinase or a molecule to which it binds is anchored to a solid phase) or a homogeneous format (where the entire reaction is carried out in a liquid phase). In either approach, the order in which the reactants are added can be varied to obtain different information about the agents being tested. For example, exposing the NDR1 or NDR2 kinase to the test agent and a binding partner at the same time identifies agents that interfere with binding (by, e.g., competition), whereas adding the test agent after binding has occurred identifies agents capable of disrupting preformed complexes (such agents may have higher binding constants and thereby displace one of the components from the complex).

Whether the methods are carried out in vitro or in vivo, they can employ biological samples. Generally, the biological sample can be provided or obtained from a test subject and can be (or can include) an organ, tissue, cell or biological fluid (e.g., a blood or serum sample) in which NDR1 or NDR2 kinase are normally expressed. The sample can be tested for NDR1 or NDR2 expression (e.g., mRNA or protein expression), structural integrity (e.g., full-length or C-terminally truncated) or for kinase activity. In vitro techniques for detecting NDR1 or NDR2 kinases include enzyme linked immunosorbent assays (ELISAs), immuno-precipitations, immunofluorescence, enzyme immunoassay (EIA), radioimmunoassay (RIA), and Western blot analysis. In vivo techniques can be carried out with labeled probes, such as anti-NDR1 or NDR2 kinase antibodies, which can be detected by standard imaging techniques. Regardless of the precise context in which NDR1 or NDR2 kinase expression is assessed, the antibodies used can be polyclonal, or more preferably, monoclonal. An intact antibody, or a fragment thereof (e.g., an Fab or F(ab′)₂ fragment) can be used. The term “labeled” is intended to encompass entities (e.g., probes such as antibodies) that are directly labeled by being linked or coupled (i.e., physically linked) to a detectable substance as well as entities that are indirectly labeled by virtue of being capable of reacting with a detectable substance or participating in a reaction that gives rise to a detectable signal.

To determine the activity of an NDR1 or NDR2 kinase, any standard assay for protein phosphorylation can be carried out. One can use a natural NDR1 or NDR2 substrate or another protein or peptide that NDR1 or NDR2 phosphorylates. For example, the NDR1 kinase can phosphorylate the peptide KKRNRRLSVA (U.S. Pat. No. 6,258,776; SEQ ID NO:6). Assays for NDR1 or NDR2 kinase activity can also be carried out with biologically active fragments of the kinase (e.g., a fragment that retains catalytic activity or, where the activity has an effect on retroviral replication or cytopathogenicity, a fragment that interacts with a retroviral protein or other component of a retrovirus). More specifically, a screen (e.g., a high throughput screen) for NDR1 or NDR2 kinase inhibitors and agonists can be carried out by: (a) binding one or more types of substrate proteins or peptides to a solid support (e.g., the wells of microtiter plates); (b) exposing the substrate to a blocking agent (standard blocking agents are known); and (c) exposing the substrate to an NDR1 or NDR2 kinase, a source of phosphate (e.g., ATP with a radioactively labeled gamma-phosphate), and a test compound (i.e., a potential NDR1 or NDR2 kinase inhibitor or agonist). The components of the reaction (e.g., the NDR1 or NDR2 kinase, phosphate source, and test compound) are typically supplied in a buffered solution and the reaction is allowed to proceed at a temperature (the temperature can vary from, for example, room temperature (about 23° C.) to a physiological temperature (about 37° C.)) and for a period of time that is in the linear range of the assay. The reaction can be terminated in a number of ways (by, for example, rinsing the support several times with a buffered solution), and the amount of phosphate incorporated into the bound substrate can be determined (standard techniques are available to measure, for example, radioactive tags). Inhibitors are identified as the agents that reduce the extent to which the NDR kinase was able to phosphorylate the substrate. Agonists are identified as the agents that increase the extent to which the NDR kinase was able to phosphorylate the substrate. See, also, U.S. Pat. No. 6,258,776 for descriptions of other assays that can be used to measure the activity of NDR1 kinases or a change in the molecules with which an NDR1 kinase interacts (e.g., the binding between an NDR1 kinase and an EF hand-containing calcium binding protein) and U.S. Pat. No. 4,109,496, which utilizes an approach in which a fluorescent label is quenched when two entities participate in a complex.

Appropriate controls can be carried out in connection with any of the methods of the invention. For example, the method described above (and others aimed at identifying NDR1 or NDR2 kinase inhibitors and agonists) can be carried out in the presence and absence of a test compound (representing experimental and control paradigms, respectively). Alternatively, test compounds and placebos (e.g., biologically inactive test compounds, such as denatured or mutant proteins or nucleic acids that lack biological activity) can be used.

The agents tested for inhibitory activity can be those within a library, and the screen can be carried out using any of the numerous approaches used with combinatorial libraries. One can use, for example, biological libraries or peptoid libraries, which contain molecules having the functionalities of peptides, but with novel, non-peptide backbones that are resistant to enzymatic degradation but which nevertheless remain bioactive (see, e.g., Zuckermann et al., J. Med. Chem. 37:2678-85, 1994). One can also use spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997). Molecular libraries can be synthesized according to methods known in the art (see, e.g., DeWitt et al., Proc. Natl. Acad. Sci. USA 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994).

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc. Natl. Acad. Sci. USA 89:1865-1869, 1992) or on phage (Scott and Smith, Science 249:386-390, 1990; Cwirla et al., Proc. Natl. Acad. Sci. 87:6378-6382, 1990; Felici, J. Mol. Biol. 222:301-310, 1991; Ladner supra.). Regardless of the precise mode of presentation, the agents in the libraries are exposed to an NDR kinase and a substrate; here, as above, agents within the libraries can be identified as inhibitors by virtue of their ability to prevent, to any extent, the ability of the kinase to phosphorylate its substrate.

NDR1 or NDR2 kinase activity can also be assayed in cell-based systems. These methods can be carried out by, for example, contacting a cell that expresses an NDR1 and/or NDR2 kinase protein, or a biologically active portion thereof, with a test agent and assessing the ability of the test agent to inhibit or activate NDR1 or NDR2 kinase activity (any assay to examine NDR1 or NDR2 kinase activity can be carried out with a biologically active portion of the whole kinase). The inhibitor can affect NDR1 or NDR2 directly or indirectly (by inhibiting or activating a molecule that acts on, or that is acted on by, NDR1 or NDR2 kinases). The agonist can affect NDR1 or NDR2 directly or indirectly (by inhibiting or activating a molecule that acts on, or that is acted on by, NDR1 or NDR2 kinases). Cell-based systems can also be used to identify agents that inhibit NDR1 or NDR2 kinase by inhibiting its expression (in that event, it is expected that the test agents will be nucleic acids (e.g., siRNA or antisense oligonucleotides) or transcription factor-binding factors, although the invention is not so limited). The cell can be any biological cell that expresses an NDR1 or NDR2 kinase, whether naturally or as a result of genetic engineering. For example, the cell can be a mammalian cell, such as a murine, canine, ovine, porcine, or human cell. The cell can also be non-mammalian (e.g., a Drosophila cell). The cell can be compared to a cell that expresses a small-interfering RNA (siRNA) that inhibits NDR1 or NDR2 kinase expression e.g. See, e.g., Devroe and Silver, BMC Biotech. 2:15, 2002, in which HeLa cell lines expressing an siRNA that interferes with NDR1, an siRNA to an unrelated protein, p75, and to other control sequences, are described.

In addition to, or as an alternative to, assessing kinase activity, the assays performed in the methods of the invention can reveal whether a test agent interferes with the ability of an NDR1 or NDR2 kinase to simply bind to, or otherwise associate with, another molecule or moiety. For example, one can determine whether a test agent inhibits the ability of an NDR1 or NDR2 kinase to bind to a substrate or a component of a retrovirus. These methods can be carried out by, for example, labeling either the NDR1 or NDR2 kinase or its binding partner (e.g., an NDR1 or NDR2 kinase substrate or a retrovirus or a component thereof) with a marker, such as a radioisotope or enzymatic label, so that NDR1 or NDR2 kinase-containing moieties (e.g., protein complexes or retroviruses that contain NDR1 or NR2) can be detected. Suitable labels are known in the art and include, for example, ¹²⁵I, ³⁵S, ¹⁴C, or ³H (which are detectable by direct counting of radioemmissions or by scintillation counting). Enzymatic labels include horseradish peroxidase, alkaline phosphatase, and luciferase, which are detected by determining whether an appropriate substrate of the labeling enzyme has been converted to product. Fluorescent labels can also be used. Another way to detect interaction (between any two molecules (e.g., an NDR1 or NDR2 kinase and an inhibitor, substrate, or retrovirus)) using a fluorophore is by fluorescence energy transfer (FET) (see, e.g., Lakowicz et al., U.S. Pat. No. 5,631,169 and Stavrianopoulos et al., U.S. Pat. No. 4,868,103). A fluorophore label on the first, or “donor,” molecule emits fluorescent energy that is absorbed by a fluorescent label on the second, or “acceptor,” molecule, which fluoresces due to the absorbed energy (the labels on the two molecules emitting different, and therefore distinguishable, wavelengths of light). Alternately, the “donor” protein can simply utilize the natural fluorescent energy of tryptophan residues. Since the efficiency of energy transfer between the labels is related to the distance separating them, the spatial relationship between the molecules can be assessed. Where the two molecules bind one another, emission from the acceptor molecule is maximal; emission can be measured readily (with, for example, a fluorimeter).

Binding can also be detected without using a labeled binding partner. For example, a microphysiometer can be used to detect the interaction of a protein or virion with NDR1 or NDR2 kinases without the labeling the protein, virion, or kinases (McConnell et al., Science 257:1906-1912, 1992). Another label-free option is to assess interaction between an NDR1 or NDR2 kinase and a target molecule (be it a kinase substrate, other binding protein, or retroviral component) with real-time Biomolecular Interaction Analysis (BIA) (see, e.g., Sjolander and Urbaniczky, Anal. Chem. 63:2338-2345, 1991 and Szabo et al., Curr. Opin. Struct. Biol. 5:699-705, 1995). BIA detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that indicates real-time reactions between biological molecules.

As noted above, NDR1 or NDR2 kinase inhibitors and agonists can be detected in assays where an NDR1 or NDR2 substrate is bound to a solid support. More generally, wherever NDR-related binding is assessed (whether between NDR1 or NDR2 and a substrate or other entity (e.g., an NDR1 or NDR2 kinase inhibitor or retroviral component)), one of the binding partners can be anchored to a solid phase (e.g., a microtiter plate, a test tube (e.g., a microcentrifuge tube) or a column). The non-anchored binding partner can be labeled, either directly or indirectly, with a detectable label (including any of those discussed herein), and binding can be assessed by detecting the label. If desired, the NDR1 or NDR2 kinase (or a biologically active fragment thereof) can be fused to a protein that binds a matrix. For example, one can identify an NDR kinase inhibitor by fusing an NDR kinase (or a potential NDR-binding partner) to glutathione-5-transferase; absorbing the fusion protein to a support (e.g., glutathione sepharose beads (Sigma Chemical, St. Louis, Mo.) or glutathione-derivatized microtiter plates); exposing the immobilized fusion protein to a potential binding partner (e.g., an agent that inhibits the activity of the kinase; i.e., a test compound); washing away unbound material; and detecting bound material. The exposure should take place under conditions conducive to complex formation (e.g., a physiologically acceptable condition). Alternatively, the complexes can be dissociated from the matrix, and the level of NDR kinase binding or activity can be determined using standard techniques.

NDR kinases or molecules with which they interact (e.g. NDR substrates) or which with they may interact (e.g., potential inhibitors) can also be immobilized on matrices using biotin and avidin or streptavidin. For example, biotinylated NDR kinases or molecules to which they bind can be prepared from biotin-NHS(N-hydroxy-succinimide) using techniques known in the art (e.g., using the biotinylation kit sold by Pierce Chemicals, Rockford, Ill.), and immobilized in the wells of avidin- or streptavidin-coated 96 well plates (Pierce Chemical). Regardless of the precise way in which an NDR kinase is immobilized, the kinase is exposed to a potential binding partner, any unreacted components are removed (e.g., by washing; under conditions that retain any complexes); and the remaining complexes are detected (by virtue of a label or with an antibody (e.g., an antibody that specifically binds the NDR kinase used in the assay). Although somewhat more labor intensive, the step of detecting an NDR kinase (or an NDR-containing protein complex) can also be carried out by enzyme-linked assays, which rely on detecting an enzymatic activity associated with the kinase or its target molecule.

Where the binding assay is carried out in a liquid phase, the reaction products (e.g., NDR-containing complexes) can be separated from unreactive components by, for example: differential centrifugation (see, e.g., Rivas and Minton, Trends Biochem. Sci. 18:284-287, 1997); chromatography (gel filtration chromatography, ion-exchange chromatography); electrophoresis (see, e.g., Ausubel et al, Eds. Current Protocols in Molecular Biology 1999, J. Wiley & Sons, New York.); and immunoprecipitation (as described, for example, in Ausubel, supra). Where FET is utilized (see above), further purification is not required.

NDR kinase modulators can also be identified by using an NDR kinase as a “bait protein” in a two- or three-hybrid assay (see, e.g., U.S. Pat. No. 5,283,317; Zervos et al., Cell 72:223-232, 1993; Madura et al., J. Biol. Chem. 268:12046-12054, 1993; Bartel et al., Biotechniques 14:920-924, 1993; Iwabuchi et al., Oncogene 8:1693-1696, 1993; and WO 94/10300). Briefly, these assays utilize two different DNA constructs; in one, the gene that codes for an NDR kinase is fused to a gene encoding the DNA binding domain of a known transcription factor (e.g., GAL-4), and in the other, a DNA sequence, from a library of DNA sequences, that encodes an unidentified protein (“prey” or “sample”) is fused to a gene that codes for the activation domain of the known transcription factor. Alternatively, the NDR kinase can be the fused to the activator domain. If the “bait” and the “prey” proteins interact, in vivo, forming a NDR kinase-dependent complex, the DNA-binding and activation domains of the transcription factor are brought into close proximity, allowing transcription of a reporter gene (e.g., lacZ) that is operably linked to a transcriptional regulatory site responsive to the transcription factor. Expression of the reporter gene can be detected and cell colonies containing the functional transcription factor can be isolated and used to obtain the cloned gene (i.e., the gene encoding the protein that interacts with the NDR kinase).

Where NDR kinase expression is assessed, a cell or cell-free mixture is contacted with a candidate compound and the expression of NDR kinase mRNA or protein is evaluated (the level can be compared to that of NDR kinase mRNA or protein in the absence of the candidate compound or in the presence of another control substance (e.g., where the candidate compound is an antisense oligonucleotide, the “control” can include a “sense” oligonucleotide)). Clearly, where mRNA or protein expression is less (statistically significantly less) in the presence of the candidate compound than in its absence, the candidate compound is an inhibitor of NDR kinase mRNA or protein expression. The level of NDR kinase mRNA or protein expression can be readily determined using methods well known in the art (e.g., Northern blot analysis, Western blot analysis or other immunoassay, by polymerase chain reaction analyses (e.g., rtPCR; see U.S. Pat. No. 4,683,202), probe arrays, and by serial analysis of gene expression (see U.S. Pat. No. 5,695,937)).

The level of mRNA corresponding to an NDR kinase gene in a cell can be determined both by in situ and by in vitro formats. Where a nucleic acid molecule is used as a probe, the probe can be, or can include, SEQ ID NO:1, or a portion thereof, such as an oligonucleotide of at least 7, 15, 30, 50, 100, or more nucleotides or ranges between (e.g., 8-14, 16-29, 31-49, or 51-99 nucleotides). The probe can be disposed on an address of an array (e.g. a two-dimensional gene chip array), which can be used in an assay to detect NDR kinase inhibitors, which can, in turn, be used as therapeutic agents (e.g., anti-retroviral agents). For in situ methods, a cell or tissue sample can be prepared and immobilized on a support, typically a glass slide, and then contacted with a probe that can hybridize to mRNA that encodes the NDR kinase gene being analyzed.

Moreover, any of the methods described above can be carried out in concert with any other(s). For example, an NDR kinase inhibitor can be identified using a cell-based or a cell free assay, and the ability of the agent to modulate the activity of a NDR kinase protein can be confirmed in vivo (e.g., in an animal such as a mouse or a non-human primate). Moreover, as described further below, one can combine (or sequentially perform) assays to identify NDR kinase inhibitors with those to identify anti-retroviral agents.

Viral Screening Assays. The invention also provides methods for identifying agents, including those determined to be NDR1 or NDR2 kinase modulators, that effect retroviral cytopathogenicity. These methods can be carried out by measuring (or qualitatively assessing) the cytotoxicity of HIV-1 on target cells (e.g., CD4+ cells, such as HeLa-CD4 cells) in the presence of a test agent (e.g., an NDR1 kinase inhibitor).

Retroviruses are classified as such because they contain an RNA genome and reverse transcriptase activity. Many classes of retroviruses have been identified, and any of these can be used in the screening methods of the invention (additional viral-related methods, including methods of treating patients who have been, or who are at risk of being, infected with a retrovirus are discussed below). For example, one can assess the ability of an NDR1 kinase inhibitor to reduce the cytopathogenicity of any of the T-lymphotropic viruses, which include HTLV-I (the apparent causative agent of adult T-cell leukemia-lymphoma), HTLV-II (the apparent causative agent of some types of hairy cell leukemia), and HIV-1 and HIV-2 (the apparent causative agents of Acquired Immune Deficiency Syndrome (AIDS)).

Assays to determine cytopathogenicity are known, and can be used in the methods of the present invention. Where an assay requires determining cell viability, that can be determined, for example, by visual inspection of cells, by staining cells with a vital dye such as 3-(4,5-Dimethyl-2-thiazolyl)-2,5-diphenyl-2H tetrazoliumbromide (MTT), by measuring cell proliferation, or by using agents that detect cellular changes characteristic of apoptotic or necrotic cells.

Endogenous retroviruses, which can also be used in the screening assays described here, are retroviruses that have integrated into the genome of the host. Reactivation of endogenous retroviruses has been linked to a variety of chronic diseases, including multiple sclerosis, Sjögren's syndrome, systemic lupus erythematosis, insulin-dependent diabetes mellitus, congenital heart block, and primary biliary cirrhosis (see, e.g., Portis, Virol. 296:1-5, 2002; Monteyne et al., Curr. Opin. Neurol. 11(4):287-91, 1998; Bing et al., J. Mol. Cell. Cardiol. 30(7):1257-62, 1998). As discussed further below, NDR1 or NDR2 modulators that have anti-retroviral activity can be used to treat patients who are diagnosed with, or who are at risk for, a disease associated with an endogenous retrovirus. Accordingly, patients amenable to treatment with an NDR kinase inhibitor include patients diagnosed with, or at risk for, multiple sclerosis, Sjögren's syndrome, systemic lupus erythematosis, insulin-dependent diabetes mellitus, congenital heart block, and primary biliary cirrhosis. While methods of treatment are described further below, we note here that treatment includes administration of a therapeutically effective amount of an NDR kinase inhibitor (e.g., an siRNA that mediates NDR kinase-specific RNAi).

The ability of an agent, which may be an agent previously identified as an NDR1 or NDR2 kinase modulator, to impede the life cycle of a retrovirus or otherwise effect its pathogenicity can be carried out by including the agent in any model system in which retroviral replication, infectivity, or pathogenicity can be assessed. Infectivity can be measured in an assay that measures, for example, the expression of viral proteins; to aid detection, these assays can employ recombinant retroviruses that express a reporter protein such as β-galctosidase, green fluorescent protein (GFP), luciferase, or the like (see, e.g., Jacque et al., Nature. 418(6896):435-8, 2002; Chen et al., J. Virol. 72(6):4765-74, 1998; Willey et al., J. Virol. 62(1):139-47, 1988, U.S. Pat. No. 6,323,019).

The anti-retroviral activity of an NDR kinase inhibitor can also be assessed by measuring reverse-transcriptase (RT) activity of viruses. Methods in which RT activity is assessed can include the steps of: growing virus in host cells in the presence and absence of a potential anti-viral agent (e.g., an NDR kinase inhibitor; once a standard is established, the assay may be conducted without growing virus in the presence of the test agent (one can, instead, simply compare the results to a previously established reference standard)); collecting culture supernatant from the host cells and isolating virions; and either measuring virion-associated RT activity directly, or infecting naïve cells with the isolated virus and measuring RT activity in the infected cells. RT activity in a sample is typically measured by incubating an RNA template, a DNA primer (or a poly(rA)-olig(dT) homopolymer template-primer), a mixture of nucleoside triphosphates, at least a portion of which carry a detectable label, and other substances to support the reaction; labeled DNA produced by the reaction is then quantitated.

Gene Profiles and Arrays: Any of the samples used in the assays of the invention can be evaluated for more than just NDR kinase expression or activity (i.e., NDR kinase expression or activity can be evaluated in the context of the expression or activity of other genes, such as retroviral genes, in the context of a gene profile). The methods in which numerous genes are evaluated can be carried out by providing a sample (e.g., a sample as described above (which may be supplied by the patient or a person who cares for the patient)) and determining the level of expression of two or more genes (e.g., 5, 10, 12, 15, 20, or 25 or more genes) in the sample, one of which is a gene that encodes an NDR kinase (other candidate genes include those that encode molecules that act upstream or downstream of the NDR kinase). The levels of expression obtained from a particular sample or subject can be compared to a reference value or reference profile, which can be obtained by any of the methods described herein (i.e., by any of the assays for DNA or protein expression or activity). Methods in which NDR kinase expression or activity is measured (whether alone or in the context of a larger gene profile) can be used to monitor a treatment for a disorder, such as a disorder caused by a retrovirus, in a subject, and the information gained can be used to adjust the subject's treatment accordingly (to bring the subject's NDR kinase expression and gene profile closer to that of a healthy individual or an individual whose treatment has been successful in reducing the signs or symptoms of the retroviral disease; see, e.g., Golub et al., Science 286:531, 1999).

Accordingly, the invention features methods of evaluating an NDR kinase, e.g., NDR1 or NDR2 kinase, or a modified form thereof; see Example 4) in a subject in order to assess the risk of, or the extent of, disease (e.g., retroviral disease) in the subject (when carried out over time, these methods can indicate the pace of the disease or the subject's responsiveness to a given treatment). The methods can be carried out by providing a biological sample from a subject and determining the level of NDR kinase expression or activity, optionally while determining the level of expression or activity of other genes. The sample can be processed (e.g., cells can be lysed and mRNA or proteins can be isolated (although absolute purity is not required); if desired, nucleic acids can be amplified) from other cellular components, and the processed sample can be applied to the array. One can then determine which addresses become occupied (by detecting array-bound nucleic acids or proteins). This reflects the nucleic acid or protein content of the sample. One can then, if desired, compare the subject's expression profile to one or more reference profiles and select the reference profile most similar to the subject reference profile (as the status of the patient providing the reference profile can be determined, a patient having a similar profile is likely to have a similar clinical status or expected course of disease).

Just as simple assays for NDR expression or activity can be carried out to identify NDR kinase inhibitors, arrays can be used to determine the effect of a potential inhibitor on NDR kinase and other genes or gene products. For example, one can treat a cell (in culture or in vivo (e.g., in an animal model)), process the cellular material to obtain mRNA or protein and apply that mRNA or protein to an array. The effect of the potential inhibitor on the sample (as evidenced by detectable binding at particular addresses of the array) indicates whether the potential inhibitor should be developed further as a therapeutic agent and, if so, what other measures should be considered. For example, if a potential inhibitor has an undesirable effect on the treated cell or another cell type, one could co-administer a counteracting agent or otherwise treat the undesired effect. Similarly, even within a single cell type, undesirable biological effects can be determined at the molecular level. Thus, the effects of an agent on expression of genes other than the target gene can be ascertained and counteracted.

As noted above, NDR kinase expression or activity, alone or in the context of other genes, can be tested in a variety of cell types to examine tissue specific expression. If a sufficient number of diverse samples are analyzed, clustering (e.g., hierarchical clustering, k-means clustering, Bayesian clustering and the like) can be used to identify other genes that are co-regulated with NDR kinase. Thus, where the methods of the invention employ arrays, they can result in quantitation of the expression of multiple genes. Quantitative data can be used to group (e.g., cluster) genes on the basis of their tissue expression per se and on their level of expression in that tissue.

A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The methods described above, in which an NDR kinase is assessed in the context of a gene profile, can be carried out with arrays, which include a substrate having a plurality of addresses, at least one of which includes a capture probe that specifically binds an NDR kinase molecule (e.g., a NDR kinase nucleic acid or a NDR kinase polypeptide). The substrate can be a glass slide, a wafer (e.g., silica, plastic or other synthetic wafer), a mass spectroscopy plate, or a three-dimensional matrix, such as a gel pad. The substrate can be densely arrayed, having at least 10, 50, 100, 200, 500, 1,000, 2,000, 5,000 or 10,000 or more addresses/cm², or any number ranging between these (e.g., 10-50, 50-100, 100-200, etc.). However, the array need not be so complex to yield useful information (i.e., fewer than a dozen or so molecules can be arrayed).

At least one address of the plurality (and, in some cases, a subset of the plurality) will include a nucleic acid capture probe that hybridizes specifically to an NDR kinase nucleic acid (the sense or anti-sense strand). Where there are a subset of NDR kinase probes, each address of the subset can include a capture probe that hybridizes to a different region of an NDR kinase nucleic acid. Alternatively, each address of the array or a subset of the plurality can include a unique polypeptide (e.g., an antibody (e.g., a monoclonal antibody or a single-chain antibody) or substrate), at least one address being capable of specifically binding an NDR kinase or a fragment (e.g., a biologically active fragment) thereof. Methods of producing polypeptide arrays are described in, for example, De Wildt et al., Nature Biotech. 18:989-994, 2000; Lueking et al., Anal. Biochem. 270:103-111, 1999; Ge, Nucleic Acids Res. 28:e3, I-VII, 2000; MacBeath and Schreiber, Science 289:1760-1763, 2000; and WO 99/51773A1. See also U.S. Pat. Nos. 5,143,854, 5,510,270, and 5,527,681, which describe arrays generated by photolithographic methods; U.S. Pat. No. 5,384,261, which describes arrays generated by mechanical methods (e.g., directed-flow methods); U.S. Pat. No. 5,228,514, which describes arrays generated by pin-based methods; and PCT application No. US/93/04145, which describes arrays generated by bead-based techniques.

Where the array includes an NDR kinase, it can be used to detect an NDR kinase-binding compound (e.g., an antibody or NDR kinase-binding protein or substrate) in a sample from a subject. Where nucleic acids are arrayed, they can be identical to an NDR kinase nucleic acid, but they need not be; they can also be homologous (having, for example, at least 60, 70, 80, 85, 90, 95 or 99% identity to an NDR kinase nucleic acid or fragment thereof (e.g., an allelic variant, site-directed mutant, random mutant, or combinatorial mutant)). Calculations of homology or sequence identity between sequences (the terms are used interchangeably herein) are performed as follows.

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. The percent identity between two nucleotide sequences can be determined using the algorithm of Needleman and Wunsch ((1970) J. Mol. Biol. 48:444-453) algorithm which has been incorporated into the GAP program in the GCG software package, using either a Blossum 62 matrix and a gap weight of 12, a gap extend penalty of 4, and a frameshift gap penalty of 5.

Generally, to determine the percent identity of two nucleic acid sequences, the sequences are aligned for optimal comparison purposes (e.g., gaps can be introduced in one or both of a first and a second nucleic acid sequence for optimal alignment and non-homologous sequences can be disregarded for comparison purposes). The length of a reference sequence aligned for comparison purposes can be at least 30%, 40%, 50%, 60%, 70%, 80%, 90%, 100% of the length of the reference sequence. The nucleotides at corresponding nucleotide positions are then compared. When a position in the first sequence is occupied by the same nucleotide as the corresponding position in the second sequence, then the molecules are identical at that position (as used herein nucleic acid “identity” is equivalent to nucleic acid “homology”).

The nucleic acid sequences described herein can be used as a “query sequence” to perform a search against NDR sequences, for example. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to evaluate identity at the nucleic acid level. BLAST protein searches can be performed with the XBLAST program, score=50, wordlength=3 to evaluate identity at the protein level. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. Software for performing BLAST analyses is publicly available through the National Center for Biotechnology Information. Alignment of nucleotide sequences for comparison can also be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Any of the methods of the invention in which NDR kinase expression or activity is assessed can include a further step whereby the result is transmitted to a caregiver or other interested party (e.g., the patient). The result can be simply the level of NDR kinase expression or activity; the level of expression or activity within the context of an expression profile; a result obtained by comparing the subject's NDR kinase or an NDR kinase-inclusive expression profile with that of a reference profiles, a most similar reference profile, or a descriptor of any of the aforementioned. The result can be transmitted in any way information travels (e.g., across a computer network by way of, for example, a computer data signal embedded in a carrier wave).

Computer media: The invention also features a computer medium having a plurality of digitally encoded data records. Each data record includes a value representing the level of expression of NDR kinase in a sample, and a descriptor of the sample. The descriptor of the sample can be an identifier of the sample, a subject from which the sample was derived (e.g., a patient), a diagnosis, or a treatment (e.g., a preferred treatment). The data record can further include values representing the level of expression of genes other than NDR kinase (e.g., other genes associated with a NDR kinase-disorder, or other genes on an array). The data record can be structured as a table (e.g., a table that is part of a database such as a relational database (e.g., a SQL database of the Oracle or Sybase database environments)).

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of NDR kinase expression RNA interference. “RNA interference” (RNAi) is a term used to refer to the mechanism by which a particular mRNA is degraded in host cells. To inhibit an mRNA, double-stranded RNA (dsRNA) corresponding to a portion of the gene to be silenced (here, NDR kinase) is introduced into a cell. The dsRNA is digested into 21-23 nucleotide-long duplexes called short interfering RNAs (or siRNAs), which bind to a nuclease complex to form what is known as the RNA-induced silencing complex (or RISC). The RISC targets the homologous transcript by base pairing interactions between one of the siRNA strands and the endogenous mRNA. It then cleaves the mRNA about 12 nucleotides from the 3′ terminus of the siRNA (see Sharp et al., Genes Dev. 15:485-490, 2001, and Hammond et al., Nature Rev. Gen. 2:110-119, 2001). RNAi has proven successful in human cells, including human embryonic kidney and HeLa cells (see, e.g., Elbashir et al., Nature 411:494-498, 2001). Gene silencing can be induced in mammalian cells by enforcing endogenous expression of RNA hairpins (see Paddison et al., Proc. Natl. Acad. Sci. USA 99:1443-1448, 2002) or, as noted above, by transfection of small (21-23 nt) dsRNA (reviewed in Caplen, Trends in Biotech. 20:49-51, 2002).

In the present invention, RNAi can be used to inhibit NDR kinase and to inhibit retrovirus replication. Various inhibitory RNAi molecules can be identified by the assays described herein (including those carried out in cell culture and those carried out in animal models of disease) and those that inhibit retroviruses can be formulated as pharmaceutical compositions to be administered in the methods of treatment discussed below.

RNAi technology utilizes standard molecular biology methods. The dsRNA (which, here, would correspond to the sequence encoding an NDR kinase) can be produced by standard methods (e.g., by simultaneously transcribing both strands of a template DNA corresponding to an NDR kinase sequence with T7 RNA polymerase; the RNA can also be chemically synthesized or recombinantly produced). Kits for producing dsRNA are available commercially (from, e.g., New England Biolabs, Inc). The RNA used to mediate RNAi can include synthetic or modified nucleotides, such as phosphorothioate nucleotides. Methods of transfecting cells with dsRNA or with plasmids engineered to make dsRNA are also routine in the art.

Gene silencing effects similar to those observed with RNAi have been reported in mammalian cells transfected with an mRNA-cDNA hybrid construct (Lin et al., Biochem. Biophys. Res. Comm. 281:639-644, 2001). Accordingly, mRNA-cDNA hybrids containing NDR kinase sequence, as well as duplexes that contain NDR kinase sequence (e.g., duplexes containing 21-23 bp monomers), are within the scope of the present invention. More specifically, the mRNA or cDNA polymers can include sequence encoding any of the 12 protein kinase catalytic subdomains identified by Hanks and Quinn (Meth. Enzymol. 200:38-62, 1991) or to the region encoding the nuclear accumulation signal. The hybrids and duplexes can be tested for anti-retroviral activity according to the assays described herein (i.e., they can serve as the test agents), and those that exhibit inhibitory activity can be used to treat patients who have, or who may develop, a disease or condition associated with retroviral infection.

The dsRNA molecules of the invention (double-stranded RNA molecules corresponding to portions of an NDR kinase gene) can vary in a number of ways. For example, they can include a 3′ hydroxyl group and, as noted above, can contain strands of 21, 22, or 23 consecutive nucleotides. Moreover, they can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand. To further enhance the stability of the RNA duplexes, the 3′ overhangs can be stabilized against degradation (by, e.g., including purine nucleotides, such as adenosine or guanosine nucleotides or replacing pyrimidine nucleotides by modified analogues (e.g., substitution of uridine 2 nucleotide 3′ overhangs by 2′-deoxythymidine is tolerated and does not affect the efficiency of RNAi). The single stranded NDR kinase RNA molecules that make up the duplex or hybrid inhibitor, or that act simply as antisense RNA oligonucleotides, are also within the scope of the invention. Any dsRNA can be used in the methods of the present invention, provided it has sufficient homology to an NDR kinase gene to mediate RNAi. While duplexes having 21-23 nucleotides are described above, the invention is not so limited; there is no upper limit on the length of the dsRNA that can be used (e.g., the dsRNA can range from about 21 base pairs of the gene to the full length of the gene or more (e.g., 50-100, 100-250, 250-500, 500-1000, or over 1000 base pairs).

When these nucleic acids are administered to a human, they can reduce NDR kinase mRNA levels and thereby treat associated retroviral disease. The cell or organism is maintained under conditions in which NDR kinase mRNA is degraded, thereby mediating RNAi in the cell or organism. Alternatively, cells can be obtained from the individual, treated ex vivo, and re-introduced into the individual.

Pharmaceutical Compositions. Modulators of NDR kinases, whether previously known or identified by the screening assays described herein, can be incorporated into pharmaceutical compositions and administered to patients who have, or who are at risk of developing, a disease associated with a retrovirus. Such compositions will include one or more inhibitors (e.g., one or more types of antisense oligonucleotides, the nucleic acid duplexes that mediate RNAi, inhibitory polypeptides (e.g., anti-NDR kinase antibodies), or synthetic agents) and a pharmaceutically acceptable carrier (e.g., a solvent, dispersion medium, coating, antibacterial and antifungal agent, isotonic and absorption delaying agent, and the like, that are substantially non-toxic). Supplementary active compounds can also be incorporated into the compositions (combination therapies are described below).

Pharmaceutical compositions are formulated to be compatible with their intended route of administration, whether oral or parenteral (e.g., intravenous, intradermal, subcutaneous, transmucosal (e.g., nasal sprays are formulated for inhalation and suppositories are formulated for vaginal or rectal administration using conventional bases such as cocoa butter and other glycerides), or transdermal (e.g., topical ointments, salves, gels, or creams as generally known in the art.)). The compositions can include a sterile diluent (e.g., sterile water or saline), a fixed oil, polyethylene glycol, glycerine, propylene glycol or other synthetic solvents; antibacterial or antifungal agents such as benzyl alcohol or methyl parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates; and isotonic agents such as sugars (e.g., dextrose), polyalcohols (e.g., manitol or sorbitol), or salts (e.g., sodium chloride). Liposomal suspensions (including liposomes targeted to infected cells with monoclonal antibodies to retroviral antigens) can also be used as pharmaceutically acceptable carriers (see, e.g., U.S. Pat. No. 4,522,811). Moreover, preparations in which NDR kinase inhibitors are so formulated and enclosed in ampoules, disposable syringes or multiple dose vials are within the scope of the invention. Where required (as in, for example, injectable formulations), proper fluidity can be maintained by, for example, the use of a coating such as lecithin, or a surfactant. Absorption of the active ingredient can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Where oral administration is intended, the NDR kinase modulator can be included in pills, capsules, troches and the like and can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Compositions containing NDR kinase modulators can be formulated for oral or parenteral administration in dosage unit form (i.e., physically discrete units containing a predetermined quantity of active compound for ease of administration and uniformity of dosage). Toxicity and therapeutic efficacy of compounds, including any potential NDR kinase modulator or anti-retroviral agent, can be determined by standard pharmaceutical procedures in cell cultures or experimental animals. One can, for example, determine the LD₅₀ (the dose lethal to 50% of the population) and the ED₅₀ (the dose therapeutically effective in 50% of the population), the therapeutic index being the ratio of LD₅₀:ED₅₀. Modulators that exhibit high therapeutic indices are preferred. Where an NDR kinase inhibitor or agonist exhibits an undesirable side effect, care should be taken to target that agent to the site of the infected tissue (the aim being to minimize potential damage to uninfected cells and, thereby, reduce side effects). Toxicity and therapeutic efficacy can be determined by other standard pharmaceutical procedures.

As is usual in drug development, the data obtained from the cell culture assays and animal studies can be used in formulating an appropriate dosage of any given NDR kinase modulator for use in humans. A therapeutically effective amount of an NDR kinase modulator will be an amount that provides an improvement in a patient's retroviral-associated disease, whether evident by improvement in an objective sign or subjective symptom of the disease. Generally, therapeutically effective amounts of proteins or polypeptide agents range from about 0.001 to 30 mg/kg body weight (e.g., about 0.01 to 25 mg/kg, about 0.1 to 20 mg/kg, or about 1 to 10 mg/kg (e.g., 2 to 9, 3 to 8, 4 to 7, or 5 to 6 mg/kg body weight). Polypeptide agents can be administered on numerous occasions (e.g., one time per week for between about 1 to 10 weeks (e.g., 2 to 8 weeks, 3 to 7 weeks, or 4, 5, or 6 weeks). One of ordinary skill in the art will understand that certain factors may influence the dosage and timing required to effectively treat a subject. These factors include, but are not limited to, the severity of the disease or disorder, previous treatments, the general health and/or age of the subject, and other diseases present.

One of ordinary skill in the art can also obtain guidance from previously performed studies. For example, antibodies (which can serve as NDR kinase modulators and anti-retroviral agents) can be delivered at a dosage of about 0.1-20 mg/kg of body weight (generally 10 mg/kg to 20 mg/kg). Where agents, such as antibodies, should affect the brain, a higher dosage (e.g., 50-100 mg/kg) may be required. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. (J. Acquired Immune Deficiency Syndromes and Human Retrovirology 14:193, 1997).

NDR kinase modulators identified and administered according to the methods of the invention can be small molecules (e.g., peptides, peptidomimetics (e.g., peptoids), amino acid residues (or analogs thereof), polynucleotides (or analogs thereof), nucleotides (or analogs thereof), or organic or inorganic compounds (e.g., heteroorganic or organometallic compounds). Typically, such molecules will have a molecular weight less than about 10,000 grams per mole (e.g., less than about 7,500, 5,000, 2,500, 1,000, or 500 grams per mole). Salts, esters, and other pharmaceutically acceptable forms of any of these compounds can be assayed and, if anti-retroviral activity is detected, administered according to the therapeutic methods described herein. Exemplary doses include milligram or microgram amounts of the small molecule per kilogram of subject or sample weight (e.g., about 1 μg-500 mg/kg; about 100 μg-500 mg/kg; about 100 μg-50 mg/kg; 10 μg-5 mg/kg; 10 μg-0.5 mg/kg; or 1 μg-50 μg/kg). While these doses cover a broad range, one of ordinary skill in the art will understand that therapeutic agents, including small molecules, vary in their potency, and effective amounts can be determined by methods known in the art. Typically, relatively low doses are administered at first, and the attending physician or veterinarian (in the case of therapeutic application) or a researcher (when still working at the clinical development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

As mentioned above, NDR modulators can include nucleic acids (e.g., nucleic acids that reduce the expression of an NDR kinase by RNAi or antisense techniques). The nucleic acid molecules can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. NDR inhibitors which target specific tissues (e.g., tissues infected with a retrovirus) can be used. For example, gene delivery vectors (e.g., viral gene delivery vectors) having a tropism for specific tissues or tissue-specific promoters can be employed to inhibit NDR kinase.

The pharmaceutical compositions can be included in a container, pack, or dispenser together with instructions for administration.

Treatment methods. As noted above, a variety of assays can be carried out to identify anti-retroviral agents, including NDR1 or NDR2 inhibitors and agonists that are effective against HIV. One can, for example, screen for agents that inhibit the NDR kinase and then test those identified as NDR inhibitors in order to determine whether they are therapeutically effective against a retrovirus (e.g., an HIV). For example, NDR inhibitors can be tested for inhibition of HIV growth on cells such as human PBMCs, other monocytic cells, or CD4⁺ HeLa cells. Alternatively, or in addition, compounds that inhibit NDR function can be tested in an animal permissive to HIV replication (e.g., the SCID-hu mouse model, U.S. Pat. No. 5,639,939, or a higher animal, such as a non-human primate). Anti-retroviral agents that continue to prove safe and effective in animal models can be tested further in human clinical trials (in, for example, HIV-positive patients).

The efficacy, toxicity, side effects, or mechanism of action, of treatment with an agent that is an NDR inhibitor can be assessed in an appropriate animal model. Furthermore, novel agents identified by the above-described screening assays can be used for treatments as described herein.

The invention provides useful methods of treating humans or non-human animals who are infected with retroviruses. Specifically, treatment of a human or animal with an effective amount of an NDR1 and/or NDR2 kinase modulator is beneficial in the treatment of retroviral infections. It is often useful to combine treatment with other anti-retroviral agents, for example protease and reverse-transcriptase inhibitors (combination therapies are discussed further below).

The cytopathogenicity of retroviruses other than HIV is also sensitive to NDR modulation, and said agents can be used to treat patients infected with such viruses. For example, feline immunodeficiency virus (FIV) infection in cats, equine infectious anemia virus (EIAV) in horses, and human T-cell leukemia virus-I and -II (HTLV-I, HTLV-II) infection in humans can be treated by modulators of the NDR kinases. The present invention provides for therapeutic as well as prophylactic methods for treating a subject at risk (or susceptible to) a disorder related to infection with a retrovirus. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

Combination therapies. NDR kinase modulators, including those identified by the methods described above, can be administered in combination with other anti-retroviral drugs, such as reverse transcriptase inhibitors, viral protease inhibitors, and viral entry inhibitors (Caliendo et al., Clin. Infect. Dis. 18:516-524, 1994). For example, one can identify a patient in need of anti-retroviral therapy (e.g., an HIV-infected subject) and administer to that patient a therapeutically effective amount of an NDR kinase inhibitor and at least one additional anti-retroviral drug. One of ordinary skill in the art can select an appropriate therapeutic regime employing one or more anti-retroviral drugs. For example, combinations and dosages of anti-retroviral drugs can be determined from published recommendations (see, e.g., Carpenter et al., J. Am. Med. Assoc. 277:1962, 1997).

More specifically, one can administer an NDR kinase inhibitor together with an inhibitor of HIV's reverse transcriptase (RT) protein, which may be either a nucleoside or non-nucleoside inhibitor of HIV RT. Examples of nucleoside RT inhibitors include, but are not limited to, zidovudine (AZT, GlaxoSmithKline), zalcitabine (ddC, Roche), didanosine (ddI, Bristol-Myers Squibb), stavudine (d4T, Bristol-Myers Squibb), abacavir (ABC, GlaxoSmithKline), tenofovir disoproxil fumarate (DF, Gilead Sciences), and lamivudine (3TC, GlaxoSmithKline). Examples of non-nucleoside RT inhibitors include, but are not limited to, efavirenz (Bristol-Myers Squibb), delavirdine (Pfizer), and nevirapine (Boehringer Ingelheim).

Alternatively, or in addition, one can administer an NDR kinase inhibitor together with an inhibitor or HIV's protease (or a combination or “cocktail” of such inhibitors). Examples of protease inhibitors include, but are not limited, saquinavir (Roche), ritonavir (Abbott), indinavir (Merck), amprenavir (Vertex/Glaxo Wellcome), nelfinavir (Agouron), and lopinavir (Abbott). Additional examples include the cyclic protease inhibitors disclosed in WO 93/07128, WO 94/19329, WO 94/22840, and the protease inhibitors disclosed in WO 94/04993, WO 95/33464, WO 96/28,418, and WO 96/28,464.

### EXAMPLES

### Example 1

### Packaging of NDR1 and NDR2 into HIV-1 Particles

We determined that NDR1 and NDR2 are incorporated into HIV-1 particles via several methodologies. Western blot analysis confirmed that NDR1 is present within lysates of sucrose-gradient purified, Optiprep velocity sedimentation purified, and subtilisin-digested preparations of HIV-1. Similarly, we detected NDR2 in sucrose-pelleted and subtilisin-digested viral preparations.

### Example 2

### NDR1 and NDR2 are Proteolytically Cleaved by the HIV-1 Protease (PR)

A fraction of the NDR1 and NDR2 present within HIV-1 lysates exhibited increased mobility following SDS-PAGE analysis, suggesting that HIV-1 expression induces post-translational modication(s) to NDR1 and NDR2. A potential HIV-1 PR cleavage site was identified near the C-terminus of NDR1 (KDWFINYT; SEQ ID NO:9) and NDR2 (KDWFLNYT; SEQ ID NO: 10). To determine whether the HIV-1 PR in fact cleaves NDR1 and NDR2, 293T cells were transiently transfected with wild-type HIV-1 (strains NL4-3 or HXBX10), or an isogenic PR-deficient strain of HXBX10, along with expression plasmids encoding epitope tagged NDR1 or NDR2 cDNAs. The faster migrating isoforms of NDR1 and NDR2 were only observed following co-expression of wild-type HIV-1, but not following co-expression of the PR-defective HIV-1 strain. These findings demonstrate that the HIV-1 PR cleaves NDR1 and NDR2.

To confirm that position 440 and 439 represent the bona fide PR-cleavage sites in NDR1 and NDR2, respectively, we constructed expression plasmids encoding epitope tagged NDR1 or NDR2 open reading frames with or without a stop codon inserted at the putative PR cleavage site. The C-terminally truncated proteins comigrated with the PR-dependent faster migrating forms of NDR1 and NDR2. In sum, HIV-1 PR-cleaved NDR1 and NDR2 kinases are observed both within purified virions and producer cell lysates. This C-terminal truncation likely alters NDR1 and NDR2 stability, subcellular localization, enzymatic activity, and/or substrate specificity. Thus, HIV-1 has evolved the ability to interact with and modify NDR1 and NDR2, suggesting these human enzymes play important role(s) in the viral life cycle.

### Example 3

### NDR1 and NDR2 Regulate HIV-1 Cytopathogenicity

To investigate the potential role of NDR1 and NDR2 in the HIV-1 viral life cycle, we down-regulated NDR1 and/or NDR2 in HeLa-CD4 cells via retrovirus-delivered RNAi (see also Devroe and Silver, BMC Biotech. 2:15, 2002). The empty vector (pMSCV/U6) and NDR1-targeting pMSCV/U6-NDR1 vector were described in Devroe and Silver, BMC Biotech. 2:15, 2002. Briefly, two oligonucleotides, Oligo 1, and Oligo 2, were synthesized.

| | | |
|---|---|---|
| Oligo 1 | 5′-GGACATGATGACCTTG | (SEQ ID NO:11) |
| (top-strand): | TTGAaagcttTCAACAAGG | |
| | TCATCATGTCCCTTTTTG- | |
| | 3′. | |
| Oligo 2 | | |
| (bottom strand): | 5′ AATTCAAAAAGGGACA | (SEQ ID NO:12) |
| | TGATGACCTTGTTGAaagc | |
| | ttTCAACAAGGTCATCATG | |
| | TCC-3′. | |

The first stretch of underlined nucleotides in Oligo 1 correspond to nucleotides 515-535 of the NDR1 open reading frame (nucleotides 1111-1130 of the NDR1 mRNA sequence, GenBank accession number NM_{—}007271; FIG. 1). The lower case letters represent a HindIII site. The second stretch of underlined nucleotides is the reverse and complement of the first. The transcribed RNA is therefore predicted to form a small hairpin. The two oligonucleotides were annealed and inserted into pBS/U6 (Sui et al., Proc. Natl. Acad. Sci. USA. 8:5515-5520, 2002) that had been digested with ApaI, treated with the Klenow fragment of DNA pol I, and digested with EcoRI, to generate pBS/U6-NDR1. pBS/U6-NDR1 was digested with BamHI to liberate the U6 promoter and the oligonucleotide region complementary to NDR. This BamHI-BamHI fragment was subsequently blunt ended with Klenow and inserted into pMSCVpuro (Clontech, Palo Alto, Calif.; vector sequence available at www.clontech.com) which had been blunt ended at the unique NsiI site, to generate pMSCV/U6-NDR1. The BamHI-BamHI fragment from pBS/U6, containing the U6 promoter and MCS, was inserted into pMSCVpuro to generate pMSCV/U6, a control puromycin-resistant vector that does not transcribe a small RNA hairpin.

To generate a retroviral RNAi vector confering Hygromycin B-resistance (pMSCVhyg/U6), pMSCVhyg (Clontech) was digested with SalI, filled in with Klenow, and digested with BglII. The fragment containing the PGK promoter and Hygromycin resistance gene was inserted into pMSCV/U6, previously digested with DraIII, filled in with Klenow, and subsequently digested with BglII. Oligonucleotides targeting nucleotides 621-641 within the NDR2 mRNA (gggtttcatccatcgggatat; SEQ ID NO:13) were inserted into pMSCVhyg/U6 essentially as described (Devroe and Silver, BMC Biotech. 2:15, 2002) except that the 3′ end of the duplex contained SalI-compatible overhangs instead of EcoRI-compatible overhangs. Retroviruses were produced as described (Devroe and Silver, BMC Biotech. 2:15, 2002), except they were packaged in 293T cells. Virus-containing supernatant was concentrated by ultracentrifugation (15,000 rpm for 3 hours in an SW28 rotor). HeLa-CD4 cells were infected as described (Devroe and Silver, BMC Biotech. 2:15, 2002), and selected in 1 μg/ml Puromycin (Clontech) and 400 μg/ml Hygromycin B (Invitrogen).

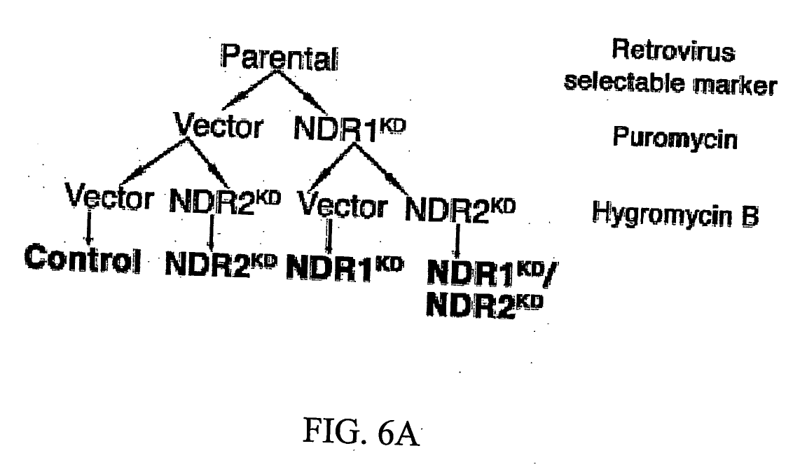

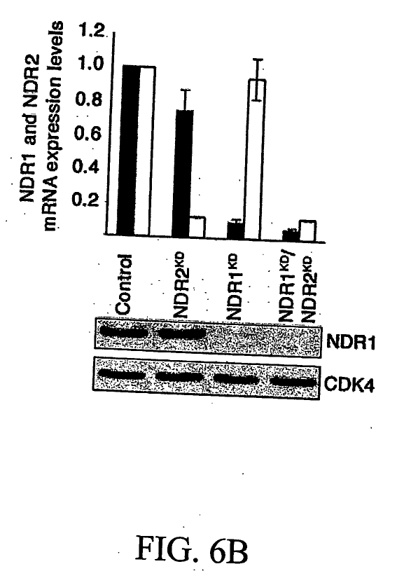

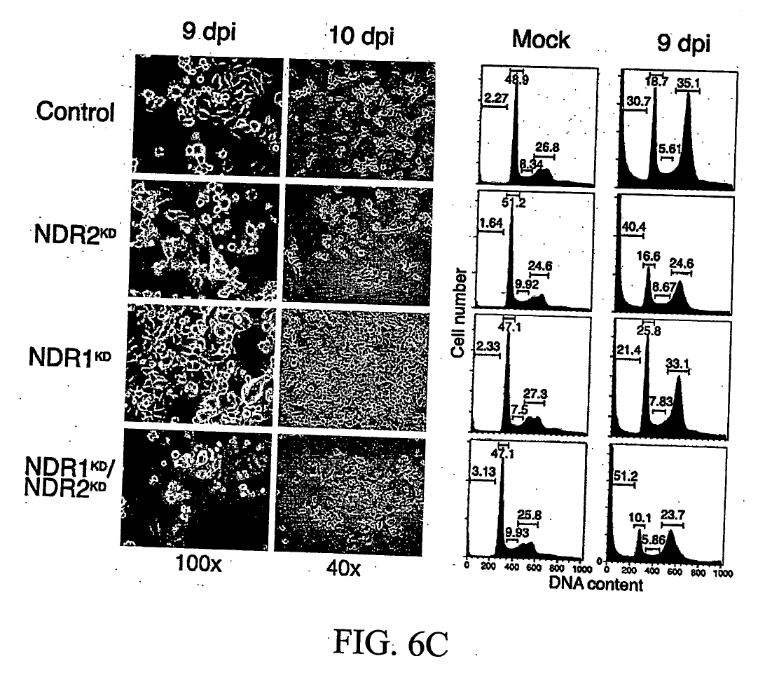

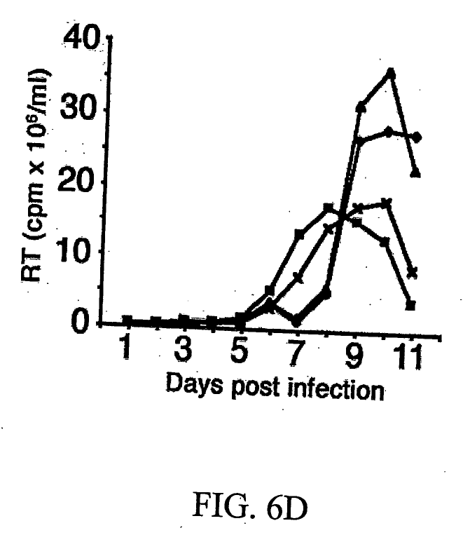

Using the protocol above, we developed “Control” HeLa-CD4 populations (infected with MSCV/U6 and MSCVhyg/U6 retroviral RNAi vectors), NDR1 knock-down cells (hereafter NDR1^{KD}; infected with MSCV/U6-NDR1 and MSCVhyg/U6), NDR2^{KD} cells (infected with MSCV/U6 and MSCVhyg-NDR2), and NDR1^{KD}/NDR2^{KD} cells (infected with MSCV/U6-NDR1 and MSCVhyg-NDR2) (FIG. 6a). Knock-down efficiency was monitored by quantitative RT-PCR on an Opticon II (MJ Research) with QuantiTect SYBR Green RT-PCR kit (Qiagen) and primers specific for NDR1 (5′-CGATGAGTTTCCAGAATCTG-3′ and 5′-GCTTGTACGTGTAATTGATG-3′; SEQ ID NO:14), NDR2 (5′-CCAGCAGCAATCCCTATAGA-3′, SEQ ID NO:15; and 5′-CAGTCTTTGGATTTGTAGTC-3′, SEQ ID NO:16), or cyclophilin A (5′-TTCATCTGCACTGCCAAGAC-3′ and 5′-TGGTCTTGCCATTCCTGGAC-3′; SEQ ID NO:17). All primer sets were designed to span a splice site within the respective genes. This analysis indicated NDR1 and NDR2 mRNA were downregulated by about 12- and 7-fold, respectively (FIG. 6b). Western blot analysis confirmed that NDR1 protein levels were similarly downregulated. Each cell line displayed indistinguishable growth rate and morphology, suggesting downregulation of NDR1 and/or NDR2 did not adversely affect normal cell physiology.

To examine the role of NDR1 and NDR2 in HIV-1 replication, the above four cell lines were seeded at 200,000 cells per well in 6-well plates and infected (in duplicate) with 10⁷ RTcpm of HIV_{NL4-3} in 1 ml of complete media. After a 4-hour incubation, cells were washed twice with serum-free DMEM and replaced with complete media. The next day, the cells were trypsinized and replated in 10-cm dishes. As early as 6 days post infection (dpi), significant cell death was observed in the NDR2^{KD} and NDR1^{KD}/NDR2^{KD} populations. Although some cytotoxicity was also observed in the NDR1^{KD} and control cell lines at 6 dpi, their rates of cell proliferation vastly exceeded cell death, requiring a backdilution of these cultures. By 9 dpi, extensive cell death was observed in NDR2^{KD} and, to an even greater extent, in NDR1^{KD}/NDR2^{KD} cell populations. In contrast, the NDR1^{KD} cells appeared significantly more resistant to HIV-1 cytopathogenicity than the control population. At the level of light microscopy, cytopathogenicity peaked between 9 and 10 dpi (FIG. 6c). At 10 dpi, the media was removed and the adherent cells were gently washed and replenished with fresh media. At this time, the NDR1^{KD} cell population approached confluence, whereas the control, NDR2^{KD} and NDR1^{KD}/NDR2^{KD} monolayers were conspicuously sparse.

Importantly, each of the mock-infected cell lines displayed indistinguishable cell cycle profiles (FIG. 6c). To quantify the differences in cell death and proliferation observed during infection, we analyzed the DNA content of each cell line at 9 dpi. At 9 dpi, floating and adherent cells were washed in PBS prior to an overnight fixation in −20° C. 95% ethanol. Fixed cells were washed in PBS containing 1% BSA, incubated with RNase A (Sigma), and stained with propidium iodide (Molecular Probes). For each cell type, DNA content of 500,000 cells was analyzed with a FacsCalibur (Becton Dickinson). Nearly 31% of the control population was dead or dying as judged by sub-G1 DNA content. Also of note, control-infected cells were largely arrested in the G2/M phase of the cell cycle, an observation consistent with known roles of HIV-1 Vpr. The NDR2^{KD} population contained significantly fewer G2/M arrested cells and a concomitant increase in cells with sub-G1 DNA content (p<10⁻¹⁵, using two-sided Normal test that approximates the binomial test). In contrast, the NDR1^{KD} population contained significantly fewer cells with a sub-G1 DNA content and a prominent increase in cells in G1 (p<10⁻¹⁵). More than half of the NDR1^{KD}/NDR2^{KD} population contained sub-G1 DNA content, with very few cells in G1.

Given that NDR1^{KD} cells are more refractory to HIV-1 cytopathogenicity, we considered that NDR1^{KD} cells might be resistant to HIV-1 infection. However, viral titers from NDR1^{KD} cell supernatants approached that of control cells (FIG. 6d). Moreover, HIV-1 did not display any defect in single-round infections of NDR1^{KD} cells. Compared to control and NDR1^{KD} cells, HIV-1 production was noticeably lower in NDR2^{KD} and NDR1^{KD}/NDR2^{KD} cells. The extensive cytopathogenicity of HIV-1 in NDR2^{KD} and NDR1^{KD}/NDR2^{KD} cells likely precluded viral production at levels comparable to that of the control population. It is worth noting that following the media change at 10 dpi, new virus production dropped precipitously in the control, NDR2^{KD}, and NDR1^{KD}/NDR2^{KD} cell lines. In contrast, NDR1^{KD} cells continued to support viral production, which indicates the NDR1^{KD} cells were both alive and productively infected with HIV-1. In sum, NDR1^{KD} cells survive in spite of HIV-1 replication, while NDR2^{KD} and NDR1^{KD}/NDR2^{KD} cells are exceptionally susceptible to HIV-1 cytopathic effects.

### Example 4

### Packaging of NDR1 into Retroviral Particles

To determine whether NDR1 is incorporated into virions of classes of retroviruses in addition to HIV, we purchased highly purified stocks of HIV-1 IIIB, HIV-2, SIVmac, EIAV (equine infectious anemia virus), and HTLV-1 from Advanced Biotechnologies, Inc. (Columbia, Md.). We determined that NDR1 was also present in each of these viruses by western blotting.

Since the all of the retroviruses listed above encode viral accessory genes (and are thus considered “complex” retroviruses), the “simple” MLV gammaretrovirus was also assayed for NDR1 incorporation. Murine NDR1 was readily detected in Rat2 producer cells; however, MLV particles did not incorporate significant quantities of NDR1. Silver staining confirmed that similar amounts of MLV and HIV-1 were analyzed. Although NDR1 was not packaged into MLV, avian NDR1 was incorporated into “simple” AMV alpharetrovirus particles, indicating that the presence or absence of viral accessory genes alone cannot predict NDR1 incorporation. Interestingly, in a majority of the viral lysates, a fraction of NDR1 exhibited altered electrophoretic mobility compared to uninfected cell lysates. This suggests that numerous retroviruses proteolytically process and incorporate NDR1. Without an antibody capable of specifically recognizing NDR2 from a variety of animal species, we cannot determine whether NDR2 is also incorporated into these retroviral particles (aside from HIV-1). However, given the presence of NDR1 in numerous classes of retroviral lysates, NDR2 is likely incorporated into many or all of these viruses as well. Thus, the NDR kinases likely regulate the cytopathogenicity of many types of retroviruses.

A number of embodiments of the invention have been described. Nevertheless, it will be understood that various modifications may be made without departing from the spirit and scope of the invention. Accordingly, other embodiments are within the scope of the following claims.

What is claimed is:

1. A method of identifying an agent that modulates an NDR kinase, the method comprising:
(a) incubating the NDR kinase with the agent under conditions that permit the agent to modulate the kinase; and
(b) performing an assay to determine the level of expression or activity of the NDR kinase, wherein a change in the level of expression or activity, relative to a control or reference standard, indicates that the agent is a modulator of the NDR kinase.

2. The method of claim 1, wherein the NDR kinase is an NDR1 kinase.

3. The method of claim 2, wherein the NDR1 kinase is a mammalian NDR1 kinase.

4. The method of claim 3, wherein the mammalian NDR1 kinase is a human NDR1 kinase.

5. The method of claim 1, wherein the NDR kinase is an NDR2 kinase.

6. The method of claim 5, wherein the NDR2 kinase is a mammalian NDR2 kinase.

7. The method of claim 6, wherein the mammalian NDR2 kinase is a human NDR2 kinase.

8. The method of claim 1, wherein the assay comprises assessing the level of NDR kinase mRNA.

9. The method of claim 8, wherein the assay comprises analysis of a Northern blot.

10. The method of claim 1, wherein the assay comprises assessing the degree to which an NDR kinase substrate has been phosphorylated.

11. The method of claim 10, wherein the substrate comprises the following amino acid sequence: KKRNRRLSVA (SEQ ID NO:6).

12. The method of claim 1, wherein the assay comprises detecting formation of a complex comprising the NDR kinase and a heterologous protein.

13. The method of claim 12, wherein the heterologous protein is a calcium binding protein.

14. The method of claim 13, wherein the calcium binding protein is an EF-hand containing calcium binding protein.

15. The method of claim 12, wherein the heterologous protein is a protein comprising an amino acid sequence at least 80% identical to a Mob protein.

16. The method of claim 1, wherein the agent decreases expression or activity of the NDR kinase, and thereby inhibits the NDR kinase.

17. The method of claim 1, wherein the agent increases expression or activity of the NDR kinase, and thereby agonizes the NDR kinase.

18. The method of claim 1, wherein the agent is a small molecule, a peptide, or a nucleic acid.

19. The method of claim 18, wherein the nucleic acid comprises a sequence that is the complement of a portion of the sequence encoding an NDR1 or NDR2 kinase.

20. The method of claim 19, wherein the nucleic acid is a double-stranded nucleic acid.

21. The method of claim 20, wherein the double-stranded nucleic acid is a small interfering RNA (siRNA).

22. The method of claim 1, wherein the NDR kinase is substantially pure.

23. The method of claim 1, wherein the NDR kinase is contained within a biological sample.

24. The method of claim 23, wherein the biological sample comprises a cell or a cellular lysate.

25. The method of claim 24, wherein the assay comprises examining the subcellular location of the NDR kinase.

26. The method of claim 24, wherein the assay comprises examining the extent to which the NDR kinase is susceptible to cleavage by a retroviral protease.

27. The method of claim 1, wherein the control is determined by conducting an assay in which the agent is omitted or supplied in an inert form.

28. The method of claim 1, further comprising examining the susceptibility of the NDR kinase to cleavage by a retroviral protease, wherein susceptibility is examined in the presence of the agent.

29. The method of claim 28, wherein the retroviral protease is an HIV-1 protease.

30. The method of claim 1, further comprising a step in which the agent that is a modulator of the NDR kinase is combined with a retrovirus-infected cell under conditions that permit the agent to modulate retroviruses produced by the cell, wherein a change in a characteristic of the retroviruses is an indication that the agent is a retroviral modulator.

31. The method of claim 30, wherein the characteristic is cytopathogenicity.

32. The method of claim 31, wherein the agent decreases cytopathogenicity of the retroviruses.

33. The method of claim 31, wherein the agent increases cytopathogenicity of the retroviruses.

34. The method of claim 30, wherein the characteristic is infectivity.

35. The method of claim 34, wherein the agent decreases infectivity of the retroviruses.

36. The method of claim 1, further comprising step (c): producing the agent identified in step (b) as a modulator of the NDR kinase.

37. A method of determining whether a modulator of an NDR kinase is also a modulator of a retrovirus, the method comprising:
(a) incubating the modulator of the NDR kinase with a retrovirus-infected cell under conditions that permit the modulator to affect retroviruses produced by the cell, and
(b) performing an assay to evaluate a characteristic of the retroviruses, wherein a change in the characteristic of the retroviruses, relative to control or a reference standard, indicates that the modulator of the NDR kinase is also a modulator of the retrovirus.

38. The method of claim 37, wherein the modulator of the NDR kinase is identified by a method comprising:
(a) contacting the NDR kinase with an agent under conditions that permit the agent to modulate the kinase; and
(b) determining whether an activity of the NDR kinase is changed in the presence of the agent, relative to a control or reference standard, wherein, if the activity of the NDR kinase is changed in the presence of the agent, the agent is identified as being a modulator of the NDR kinase.

39. The method of claim 37, wherein the modulator is a small molecule, a peptide, or a nucleic acid.

40. The method of claim 37, wherein the retrovirus is a human immunodeficiency virus-1 (HIV-1), a human immunodeficiency virus-2 (HIV-2), a human T cell leukemia virus-1 (HTLV-1), a human T cell leukemia virus-2 (HIV-2), a simian immunodeficiency virus (SIV), a feline immunodeficiency virus (FIV), or an equine infectious anemia virus (EIAV).

41. The method of claim 37, wherein the retrovirus is an endogenous retrovirus.

42. The method of claim 37, wherein the characteristic is infectivity.

43. The method of claim 42, infectivity is assessed by determining whether, following incubation of the retrovirus-infected cell with the NDR kinase modulator, the virions produced by the cell, relative to control or a reference standard: are less infectious; package less NDR kinase; contain a viral protein that is phosphorylated to a lesser extent; or exhibit less reverse transcriptase activity.

44. The method of claim 37, wherein the characteristic is an effect on the survival of cells infected with the retrovirus.

45. The method of claim 44, wherein the effect on cell survival is assessed by evaluating the survival of cells infected with the retroviruses at 5-7 days post infection, and/or 9-10 days post infection, and/or 12-18 days post infection.

46. A method of treating a subject who has been, or who is at risk of being, exposed to a retrovirus, or who has been diagnosed as having a retroviral infection or a disease associated with a retroviral infection, the method comprising, optionally, identifying the subject and administering to the subject an effective amount of a modulator of an NDR kinase.

47. The method of claim 46, wherein the NDR kinase is an NDR1 kinase.

48. The method of claim 46, wherein the NDR kinase is an NDR2 kinase.

49. The method of claim 46, wherein the modulator is an agent that inhibits the NDR kinase.

50. The method of claim 46, wherein the modulator is an NDR kinase agonist.

51. The method of claim 46, wherein the retrovirus is a lentivirus.

52. The method of claim 46, wherein the retrovirus is HIV-1, HIV-2, HTLV-1, HTLV-2, SIV, FIV, or EIAV.

53. The method of claim 46, wherein the modulator is a small molecule, a peptide, or a nucleic acid.

54. The method of claim 46, wherein the modulator comprises (a) a nucleic acid sequence encoding an NDR kinase modulator, the sequence being optimally contained with an expression vector or (b) an NDR kinase-specific siRNA.

55. The method of claim 46, wherein the modulator reduces the quantity of the NDR kinase in the host cell.

56. The method of claim 46, wherein the modulator interferes with the ability of the NDR kinase to form a complex with a retroviral protein, and/or retroviral capsids.

57. The method of claim 46, wherein the modulator inhibits the formation of a complex comprising the NDR kinase and a retroviral protease.

58. The method of claim 57, wherein the retroviral protease is an HIV protease.

59. The method of claim 46, wherein the modulator reduces the catalytic activity of the NDR kinase.

60. The method of claim 46, wherein the modulator enhances the catalytic activity of the NDR kinase.

61. The method of claim 46, wherein the inhibitor is administered in combination with at least one other anti-retroviral agent.

62. The method of claim 61, wherein the other anti-retroviral agent is a reverse transcriptase inhibitor, a viral protease inhibitor, an integrase inhibitor, or a viral entry inhibitor.

63. The method of claim 61, wherein the other anti-retroviral agent is zidovudine (AZT), lamivudine (3TC), didanosine (ddI), abacivir, zalcitabine (ddC), stavudine (d4T), tenofovir disproxil fumarate (DF), efavirenz, rescriptor, viviradine, nevirapine, delaviridine, saquinavir, ritonavir, indinavir, nelfinavir, agenerase, viracept, amprenavir, lopinavir, enfuviritide or hydroxyurea.

64. A method of determining whether a modulator of an NDR kinase is also a modulator of a retrovirus, the method comprising:
(a) transfecting a producer cell with one or more nucleic acids, wherein the nucleic acids comprise an HIV genome or a biologically active portion thereof:
(b) incubating the producer cell with the modulator under conditions that permit the modulator to affect the NDR kinase;
(c) maintaining the producer cell under conditions that allow the production of HIV virions;
(d) performing an assay to evaluate a characteristic of the virions, wherein a change in the characteristic, relative to control or a reference standard, indicates that the modulator of the NDR kinase is also a modulator of the retrovirus.

65. The method of claim 64, wherein the producer cell is transfected with two nucleic acids, wherein the first nucleic acid comprises an HIV genome that lacks a functional envelope gene; and the second nucleic acid comprises the envelope gene.

66. The method of claim 64, wherein the characteristic is cytopathogenicity.

67. The method of claim 64, wherein characteristic is infectivity.

68. The method of claim 64, wherein one or more of the transfected nucleic acids comprise a reporter gene.

69. The method of claim 67, wherein the assay comprises infecting a naïve cell with the virions and determining the activity of the reporter gene, relative to control or a reference standard.

70. The method of claim 68, wherein the reporter gene is the luciferase gene.

71. The method of claim 69, wherein the assay comprises lysing the naïve cell, after the infecting step, and measuring activity of the reporter gene.

72. The method of claim 64, wherein the assay comprises determining whether the virions package less NDR kinase, relative to control.

73. The method of claim 64, wherein the assay comprises determining whether the virions contain a viral or host protein that is phosphorylated to a lesser extent than virions produced in a control cell.

74. The method of claim 64, wherein the assay comprises determining whether the virions exhibit less reverse transcriptase activity relative to control.

75. The method of claim 64, wherein the producer cell is a HeLa cell, a T cell, or a macrophage cell.

76. The method of claim 69, wherein the naïve cell is a HeLa cell expressing a CD4 gene.

77. The method of claim 64, wherein the modulator is a small molecule, peptide, or nucleic acid.

78. The method of claim 69, wherein a plurality of producer cells are transfected, and wherein a plurality of naïve cells are infected.

79. The method of claim 46, wherein the subject has multiple sclerosis, Sjögren's syndrome, systemic lupus erythematosis, insulin-dependent diabetes mellitus, congenital heart block, and primary biliary cirrhosis.

80. The method of claim 46, wherein the subject has an acquired immune deficiency syndrome.

81. Use of an NDR kinase inhibitor in the preparation of a medicament for treating a patient who has a retroviral infection or a retroviral-associated disease.

82. Use of the NDR kinase inhibitor of claim 81, wherein the inhibitor is an siRNA that specifically inhibits NDR1 or NDR2.


SEQUENCE LISTING (19 sequences)

SEQ ID NO: 1 - DNA, 3018 bp, Homo sapiens
gaattccgggccaggcatggtagcgcatcgctgtaatcccagctactcgggaaactgaggtgggagaatcgattgaacctggaagtggaggttgcggtgagccaagatcatcctgtcgcactccagcctgggcaacaagagcgaaactccatctcaaaaagaaaaaaaaagatatatatgtgtgacttacaggtacaggtaaagttgcttctggttttctggttgttgcatggtatttcctatgcagccacaggtctttattttcttacttaagtgcctccaacttcccataacacaaattaaggcatgatgaacatcctctctgtgctgaacatcctgtgtatgtcacttcagaagcctgtgtgacggtttctttagtctttatacctaggggtgggatttctgggtcataggacagtaatttatatttatttcactaagtattctctttctctggcttttgttacatattacctgtttgtcctccagaaaacttgcaccaatttacattcctaccaatagggtaggagagtgcacaatgggtggattctaactccaaatctaacacctcttcttttctttgtttctagcagccatggcaatgacaggctcaacaccttgctcatccatgagtaaccacacaaaggaaagggtgacaatgaccaaagtgacactggagaatttttatagcaaccttatcgctcaacatgaagaacgagaaatgagacaaaagaagttagaaaaggtgatggaagaagaaggcctaaaagatgaggagaaacgactccggagatcagcacatgctcggaaggaaacagagtttcttcgtttgaagagaacaagacttggattggaagattttgagtccttaaaagtaataggcagaggagcatttggtgaggtacggcttgttcagaagaaagatacgggacatgtgtatgcaatgaaaatactccgtaaagcagatatgcttgaaaaagagcaggttggccacattcgtgcggagcgtgacattctagtggaggcagacagtttgtgggttgtgaaaatgttctatagttttcaggataagctaaacctctacctaatcatggagttcctgcctggaggggacatgatgaccttgttgatgaaaaaagacactctgacagaagaggagactcagttttatatagcagaaacagtattagccatagactctattcaccaacttggattcatccacagagacatcaaaccagacaaccttcttttggacagcaagggccatgtgaaactttctgactttggtctttgcacaggactgaaaaaagcacataggacagaattttataggaatctgaaccacagcctccccagtgatttcactttccagaacatgaattccaaaaggaaagcagaaacctggaaaagaaatagacgtcagctagccttctccacagtaggcactcctgactacattgctcctgaggtgttcatgcagaccgggtacaacaagctctgtgattggtggtcgcttggggtgatcatgtatgagatgctcatcggctacccacctttctgttctgagacccctcaagagacatataagaaggtgatgaactggaaagaaactttgacttttcctccagaagttcccatctctgagaaagccaaggatctaattttgaggttctgctgtgaatgggaacatagaattggagctcctggagttgaggaaataaaaagtaactctttttttgaaggcgttgactgggaacatatcagagagagacctgctgcaatatctattgaaatcaaaagcattgatgatacctcaaacttcgatgagtttccagaatctgatattcttaagccaacagtggccacaagtaatcatcctgagactgactacaagaacaaagactgggtcttcatcaattacacgtacaagcgctttgagggcctgactgcaaggggggcaataccttcctacatgaaagcagcaaaatagtactcttgccacggaatcctatgtggagcagagttctttgtataacatcatgcttttcctctcacactcttgaagagcttccaagaagttgatggaacccaccaatatgtcatagtaaagtctcctgaaatgtggtagtaagaggattttcttccataatgcatctgaaaaactgtaaacaaagacaaccatttctactacgtcggccataaacagctatcctgctttggaagagaagcatcatgagccaatttgataggtgttttaaaaataacttgagttttcctaagttcatcagaatgaaggggaaaaacagccatcatccaacattattgagattgtcgtgtatagtcatcgaatatcagccagttcctgtaattttgtgacacgctctctgccaagcccaccaagtatttcctttatagctaaaagttccatagtactaaggaaataaagcaataaagacagtctcagcagccaggattctggctgaaggaaatgatccgccaccctgagggtggtgatggtagtttctacccatacctcagcctcaggcgagtggcttatagcctccattcatggtgcactttatttatggtactaagataaagactgtcaatccattgatttatctcctcctgtcccccatctaaaatacccatgctgcttttctgagtgttgatgggggttaccagcttgatccactgttgctcttagaaggcccagaaagtctttgggcattgcaagaaatcccgaattatgtggaaaaccctcactttctcttcacggctgtaccagaaaatccctaagacagatcttgccgtggactagcaatacctgcaagtgctgccaatgggaactcaatttattcctgggaacctaacgaggagagcccaggcctaggcaggaggcctggaaccctcttggctaaggtgctgttcctgttcctgcaaggtctccagaacccctttggaaatggtgaaggaaccagcccaatagaagtacagagccagctgacggaattc

SEQ ID NO: 2 - protein, 465 aa, Homo sapiens
MAMTGSTPCSSMSNHTKERVTMTKVTLENFYSNLIAQHEEREMRQKKLEKVMEEEGLKDEEKRLRRSAHARKETEFLRLKRTRLGLEDFESLKVIGRGAFGEVRLVQKKDTGHVYAMKILRKADMLEKEQVGHIRAERDILVEADSLWVVKMFYSFQDKLNLYLIMEFLPGGDMMTLLMKKDTLTEEETQFYIAETVLAIDSIHQLGFIHRDIKPDNLLLDSKGHVKLSDFGLCTGLKKAHRTEFYRNLNHSLPSDFTFQNMNSKRKAETWKRNRRQLAFSTVGTPDYIAPEVFMQTGYNKLCDWWSLGVIMYEMLIGYPPFCSETPQETYKKVMNWKETLTFPPEVPISEKAKDLILRFCCEWEHRIGAPGVEEIKSNSFFEGVDWEHIRERPAAISIEIKSIDDTSNFDEFPESDILKPTVATSNHPETDYKNKDWVFINYTYKRFEGLTARGAIPSYMKAAK

SEQ ID NO: 3 - DNA, 4725 bp, Homo sapiens
agcggagagttcagggaggccgccctgagattccggcgaggccgcgggtcccacctcccgggggcggggcgagggcggagcggggagaagggagctgacgggcgcccggccggctgcggtccgtgcggaggctgagccggccgcgggcgcgaccggaggcagtttccgttactatggcaatgacggcagggactacaacaacctttcctatgagcaaccatacccgggaaagagtgactgtagccaagctcacattggagaatttttatagcaacctaattttacagcatgaagagagagaaaccaggcagaagaaattagaagtggccatggaagaagaaggattagcagatgaagagaaaaagttacgtcgatcacaacacgctcgcaaagaaacagagttcttacggctcaaaaggaccagacttggcttggatgactttgagtctctgaaagttataggaagaggagcttttggagaggtgcggttggtccagaagaaagatacaggccatatctatgcaatgaagatattgagaaagtctgatatgcttgaaaaagagcaggtggcccatatccgagcagaaagagatattttggtagaagcagatggtgcctgggtggtgaagatgttttacagttttcaggataagaggaatctttatctaatcatggaatttctccctggaggtgacatgatgacattgctaatgaagaaagacaccttgacagaagaggaaacacagttctacatttcagagactgttctggcaatagatgcgatccaccagttgggtttcatccatcgggatattaagccagacaaccttttattggatgccaagggtcatgtaaaattatctgattttggtttatgtacgggattaaagaaagctcacaggactgaattttatagaaatctcacacacaacccaccaagtgacttctcatttcagaacatgaactcaaagaggaaagcagaaacttggaagaagaacaggagacaactggcatattccacagttgggacaccagattacattgctccagaagtattcatgcagactggttacaacaaattgtgtgactggtggtctttgggagtgattatgtatgaaatgctaataggatatccacctttctgctctgaaacacctcaagaaacatacagaaaagtgatgaactggaaagaaactctggtatttcctccagaggtacctatatctgagaaagccaaggacttaattctcagattttgtattgattctgaaaacagaattggaaatagtggagtagaagaaataaaaggtcatcccttttttgaaggtgtcgactgggagcacataagggaaaggccagcagcaatccctatagaaatcaaaagcattgatgatacttcaaattttgatgacttccctgaatctgatattttacaaccagtgccaaataccacagaaccggactacaaatccaaagactgggtttttctcaattatacctataaaaggtttgaagggttgactcaacgtggctctatccccacctacatgaaagctgggaagttatgaatgaagataacattcacccataaccaagagaactcaggtagctgcatcaccaggcttgcttggcgtagataacaatacactgaaatactcctgaagatggtggtgcttattgactacaagaggaaattctacaggattaggatttctaagactactataggaattggttggcagtgccagctggctcttttttttaatattttattatttttgttaactttattatatgaaggtactggaataaaaggaacagacatccctttctaactgcactgcttacatgcgtattaaggtccattctgcctgtgtgtgctgtggctttgaactgtaacacctctaatcaattcaggaggaacacatatcatttaaagcaacataggctaacctgtaggtaacactgcagtattgatgttttactgcaaatcttatgggtctagataatcagtaaaagccatcttccatagttggtgttagaacattgccctattggtttggacatctgtagaatatatatgaagacaatttctgtaatggttttaagagatttaaaaagaaattcactggttctttacaaaatagaatttatcatcaagttattacacaaacttcacagtaaggagtgacaagtttataataaggaagacaaagtttaacaccttcactcaagcactccactaatatatttacgttgcattcagaaatactgatgaccttcatatacgtagtctgtatactcatagggagatgtactgtattatataacatgtaaagttgattttcttgtgacaagagaacttctttttttaacaagaggacatggcattattttaatttgattatggtgagttgaatttaagacatgaccatgaaggctgcttgtagaattagtgtatttttattaaactatttttttaaatgtcaaacttctatcatgtaaatggacttatagagaacaaaaagctatttactttggttttctagaaagttgttacatatcatggctggttaacttttatttcttttgatgaaaatttttcctttgatagtacttgtattattgtgccattattttcttatgctccaaatgtaccaaagatcttgaacagagtggatgttcacaactgagtagaattttcctttcctgtgggcatgctgtattcagacctgacagatctttgatagaggtcagcttattaaagggcaatattgttcttgtttagctacatcactgtggtgaatatagatggaattaaggaagtaaatgcaggccagggggttgtgatgagaggataggggagataatatcagcatcaaattctttgggtatctctctaagaattaaataatcttttctagcttaatattttaattctaattcaaacaactctgaggttttggtttcattagtaatagttgaggaataatatactagcaaagaatggcctaatgtttgtcataactgttaatggatgaaattttttaaagatacaaccatgataaccattataaatgatctatgatcaaaatctaaagtgatgaattatttgtaggaatgtcttcctaatggggaagaattgcataggagcattatgcaaatctacacaagcttttataaatgttgctgctgggtagctccacagtgtttcataaggccatcctgtttcccccaactcccccatttttggtttgtttctttttaaatatttgttgagtacttatgtgtttatctaacagttcacttccatttttctagtctggattttttgagtatttaggaaagagagctattaaaaactctggggatttctcaatgtgactaactctaatttttctaattataactgcctttaattaacataatattaacttttgctgaggtttatgagattttctcaccccacatcgctcccctttttttaaaaaggactgttttgctagtgtgataatgaataggtaagatatgagataattgcaacattgtctagttctagtatggtaactattcttgaaatggtattgaaaaataccgttaattcaaattgacagagattgataaaaagaaactgatttacctaagtttactttttaattgcataatagagcattttttgttttgagttccctcattcttattaccagaaagagcttgcaaatagttttactttcttggcactggaagggtagttctggaaagctactttgttgagagtctcattcttccctggagttaatagagtgattcacaatctttggggttttctcctcatcaaaagcatttcttaagtgcctatctaaaagcaattaaagactgtgtctgccctttagaagctaagaatttgattcatgatgcaaattaactagataatttgcaaagtacccttgagattgaattttctctattatatatttcccatatttcaggtgaataatttaatttaaatgacaaaaccctatctagtctactgggcataatgacattttctttaaattagactctattttgaattaaaagagttttattataaaccgtgtgtttttggtttttctaagtatatagaaagcttgtataattcagatttatcaatttcctgatttaatgtagactttgacttttttattaaaaacctttgtattaaagcaagttatgttatttttcttttatgcatttattactaacatagctttaaatctttaaatgtattgaagcattgtgctgtctgaaaataaggaattgcttataaaccagccacttctgaatacaatatgtagctgatttaataagctagttagtgaatggaaaataagtgtggagtattaaaaatgttctttggttggtaaggcctaagatagggtttcatttatttctatactttttctgttttttaaacacctgcatatttttatgtaaatctctaaatttaaaatattttaagtacatttatttttggtgttttattgtataaaaccttagacaatcaatcagtcagtctttactgacaggagcagcagctatctgtcttttgctgatctacaaataaatgaattgagaatttagtccatagaggtccctggctaccaaacacattctcctttgaattgttaaaattcagaacattcaaaataactgttttgctacaacccaaaaaaaaaaaaaaaa

SEQ ID NO: 4 - protein, 464 aa, Homo sapiens
MAMTAGTTTTFPMSNHTRERVTVAKLTLENFYSNLILQHEERETRQKKLEVAMEEEGLADEEKKLRRSQHARKETEFLRLKRTRLGLDDFESLKVIGRGAFGEVRLVQKKDTGHIYAMKILRKSDMLEKEQVAHIRAERDILVEADGAWVVKMFYSFQDKRNLYLIMEFLPGGDMMTLLMKKDTLTEEETQFYISETVLAIDAIHQLGFIHRDIKPDNLLLDAKGHVKLSDFGLCTGLKKAHRTEFYRNLTHNPPSDFSFQNMNSKRKAETWKKNRRQLAYSTVGTPDYIAPEVFMQTGYNKLCDWWSLGVIMYEMLIGYPPFCSETPQETYRKVMNWKETLVFPPEVPISEKAKDLILRFCIDSENRIGNSGVEEIKGHPFFEGVDWEHIRERPAAIPIEIKSIDDTSNFDDFPESDILQPVPNTTEPDYKSKDWVFLNYTYKRFEGLTQRGSIPTYMKAGKL

SEQ ID NO: 5 - protein, 12 aa, Homo sapiens
KRKAETWKRNRR

SEQ ID NO: 6 - protein, 10 aa, Homo sapiens
KKRNRRLSVA

SEQ ID NO: 7 - protein, 6 aa, Homo sapiens
DIKPDN

SEQ ID NO: 8 - protein, 9 aa, Homo sapiens
GTPDYIAPE

SEQ ID NO: 9 - protein, 8 aa, Homo sapiens
KDWFINYT

SEQ ID NO: 10 - protein, 8 aa, Homo sapiens
KDWFLNYT

SEQ ID NO: 11 - DNA, 53 bp, Artificial Sequence
  Synthetically generated oligonucleotide
ggacatgatgaccttgttgaaagctttcaacaaggtcatcatgtccctttttg

SEQ ID NO: 12 - DNA, 57 bp, Artificial Sequence
  Synthetically generated oligonucleotide
aattcaaaaagggacatgatgaccttgttgaaagctttcaacaaggtcatcatgtcc

SEQ ID NO: 13 - DNA, 21 bp, Artificial Sequence
  Targeting nucleotides
gggtttcatccatcgggatat

SEQ ID NO: 14 - DNA, 20 bp, Artificial Sequence
  Primer
gcttgtacgtgtaattgatg

SEQ ID NO: 15 - DNA, 20 bp, Artificial Sequence
  Primer
ccagcagcaatccctataga

SEQ ID NO: 16 - DNA, 20 bp, Artificial Sequence
  Primer
cagtctttggatttgtagtc

SEQ ID NO: 17 - DNA, 20 bp, Artificial Sequence
  Primer
tggtcttgccattcctggac

SEQ ID NO: 18 - DNA, 20 bp, Artificial Sequence
  Primer
cgatgagtttccagaatctg

SEQ ID NO: 19 - DNA, 20 bp, Artificial Sequence
  Primer
ttcatctgcactgccaagac